(19) United States
(12) Patent Application Publication
(10) Pub. No.: US 2016/0038459 A1
(43) Pub. Date: Feb. 11, 2016

(54) COMPOSITIONS AND METHODS FOR THE TREATMENT OF NATAL AND PRE-NATAL CONDITIONS WITH ALPHA-TOCOPHEROL

(71) Applicant: Northwestern University, Evanston, IL (US)

(72) Inventor: Joan Cook-Mills, Aurora, IL (US)

(21) Appl. No.: 14/823,117

(22) Filed: Aug. 11, 2015

Related U.S. Application Data
(60) Provisional application No. 62/035,688, filed on Aug. 11, 2014.

(51) Int. Cl. A61K 31/355; A23L 1/302; A23L 1/304; A61K 9/20; A23L 1/29

(52) U.S. Cl. CPC A61K 31/355; A61K 9/2013; A61K 9/2009; A23L 1/296; A23L 1/304; A23L 1/302; A23V 2002/00

(57) ABSTRACT

The present invention provides methods, compositions, and systems for preventing or reducing an allergic condition in an offspring by administering tocopherol to a mother pregnant or nursing the offspring, where the tocopherol in the composition is 98-100% unmodified natural d-alpha tocopherol, and less than 2% gamma tocopherol (e.g., undetectable levels of gamma tocopherol). In certain embodiments, a prenatal tablet or pill is provided composed of such tocopherol compositions in combination with folic acid, iron, and calcium.

### CROSS REFERENCE TO RELATED APPLICATIONS

The present application claims priority to U.S. Provisional Patent Application No. 62/035,688, filed Aug. 11, 2014, which is hereby incorporated by reference in its entirety.

### STATEMENT REGARDING FEDERAL SUPPORT

This invention was made with government support under R01 AT004837 and R01 HLB111624 awarded by the National Institutes of Health. The government has certain rights in the invention

### FIELD OF THE INVENTION

The present invention provides methods, compositions, and systems for preventing or reducing an allergic condition in an offspring by administering tocopherol to a mother pregnant or nursing the offspring, where the tocopherol in the composition is 98-100% unmodified natural d-alpha tocopherol, and less than 2% gamma tocopherol (e.g., undetectable levels of gamma tocopherol). In certain embodiments, a prenatal tablet or pill is provided composed of such tocopherol compositions in combination with folic acid, iron, and calcium.

### BACKGROUND

Development of allergic disease originates as complex environmental and genetic interactions that may start early in life (49), a time when the airways and the immune system are developing. In reports examining human maternal and paternal asthma associations with development of allergies in offspring, most associations are with maternal asthma (46). In animal studies, offspring from allergic mothers are predisposed to responses to suboptimal doses of allergens, and this risk is sustained to 8 weeks of age in the mouse (26, 28-30, 32, 33, 36, 46). Interestingly, in animal studies, dendritic cells (DCs) from offspring of allergic mothers transfer the risk for allergies to naïve neonates, indicating a functional change in neonatal DCs during neonate development in allergic mothers (24). It is suggested that risk for allergic disease in humans is associated with in utero and early exposures to environmental factors (9).

### SUMMARY OF THE INVENTION

The present invention provides methods, compositions, and systems for preventing or reducing an allergic condition in an offspring by administering tocopherol to a mother pregnant or nursing the offspring, where the tocopherol in the composition is 98-100% unmodified natural d-alpha tocopherol, and less than 2% gamma tocopherol (e.g., undetectable levels of gamma tocopherol). In certain embodiments, a prenatal tablet or pill is provided composed of such tocopherol compositions in combination with folic acid, iron, and calcium.

In some embodiments, provided herein are methods of preventing, or reducing the severity of, a condition in a neonate, infant, or child comprising: administering a composition to a mother of a neonate, infant, or child: 1) prior to birth of the neonate, infant, or child, and/or 2) during a time period wherein the mother is breast feeding the neonate, infant, or child; wherein the composition comprises at least 5 international units (IU's) of tocopherol (e.g., 5 . . . 100 . . . 300 . . . 500 . . . 750 . . . or 100 IU's), wherein at least 98% of all of the tocopherol in the composition is unmodified natural d-alpha-tocopherol, and wherein less than 2% of all of the tocopherol in the composition is gamma-tocopherol, and wherein the administering prevents, or reduces the severity of, an inflammatory condition in the neonate, infant, or child.

In particular embodiments, provided herein are compositions (e.g., prenatal tablet compositions) comprising: at least 5 international units (IUs) of tocopherol, wherein at least 98% of all of the tocopherol in the composition is unmodified natural d-alpha-tocopherol, and wherein less than 2% of all of the tocopherol in the composition is gamma-tocopherol.

In certain embodiments, provided herein are compositions (e.g., prenatal tablet compositions) comprising: a) at least 5 international units (IUs) of tocopherol, wherein at least 98% of all of the tocopherol in the composition is unmodified natural d-alpha-tocopherol, and wherein less than 2% of all of the tocopherol in the composition is gamma-tocopherol; b) at least 300 μg of folic acid, c) at least 20 mg of iron, and d) at least 125 mg of calcium.

In other embodiments, the composition is formulated in a pre-natal tablet or pre-natal pill (e.g. suitable for administration to a woman who is pregnant on a daily basis while she is pregnant), wherein the at least 8 IUs of tocopherol is at least 25 IUs of tocopherol, wherein the at least 300 μg of folic acid is at least 600 μg of folic acid, wherein the at least 20 mg of iron is at least 27 mg of iron, and wherein the at least 125 mg of calcium is at least 800 mg of calcium. In some embodiments, the compositions further comprise at least one of the following: 200 μg of iodine, at least 3000 IUs of vitamin A, at least 60 mg of vitamin C, at least 300 IUs of vitamin D, at least 1.25 mg of vitamin B1, at least 1.5 mg of vitamin B2, at least 15 mg of vitamin B3, at least 2.0 mg of vitamin B6, at least 3 μg of vitamin B12, at least 10 mg of zinc, at least 200 μg of biotin, at least 8 mg of pantothenic acid, and at least 2 mg of copper.

In other embodiments, provided herein are systems comprising: a) the compositions recited above and herein formulated as a tablet (e.g., prenatal table), and b) a pill comprising DHA (Docosahexaenoic acid) and/or EPA (Eicosapentaenoic acid).

In further embodiments, the condition premature fetal lung disease, full term pulmonary distress, childhood asthma, or an allergic condition (e.g., seasonal allergies, ragweed allergy, pollen allergy, dust mite allergy, food allergy, etc.). In other embodiments, the mother has at lease one type of allergy (e.g., asthma, seasonal allergy, dust mite allergy, food allergy, etc.). In further embodiments, the methods further comprise, prior to the administering, testing the mother for at lease one type of allergy (e.g., asthma, seasonal allergy, food allergy, pollen allergy, dust mite allergy, etc.). In particular embodiments, the at least 99% of all of the tocopherol in the composition is unmodified natural d-alpha-tocopherol, and wherein less than 1% of all of the tocopherol in the composition is gamma-tocopherol. In further embodiments, at least 99.9% of all of the tocopherol in the composition is unmodified natural d-alpha-tocopherol, and wherein less than 0.1% of all of the tocopherol in the composition is gamma-tocopherol. In some embodiments, the gamma-tocopherol is undetectable or nearly undetectable in the composition.

In certain embodiments, the administering to the mother is daily during the course of at least one trimester of the pregnancy of the mother (e.g., entire first, second, third, or all three trimesters in a human mother). In other embodiments, the methods further comprise, after the birth of the neonate, infant, or child, administering the composition to the neonate, infant, or child (e.g., daily for at least a week or a month or a year). In additional embodiments, the composition is administered daily to the subject for a period selected from: about 1 week, about 2 weeks, about 3 weeks, about 1 month, about 2-3 months, about 3-6 months, about 6-9 months, about 12-18 months, about 18 to 24 months, or about 24 to 30 months.

In particular embodiments, the composition comprises a prenatal pill suitable for daily administration, wherein the prenatal pill comprises: 1) the tocopherol, and 2) at least one additional ingredient selected from: A) at least 300 μg of folic acid, B) at least 20 mg of iron, and C) at least 125 mg of calcium. In other embodiments, the prenatal pill comprises the tocopherol, the folic acid, the iron, and the calcium. In other embodiments, the at least 300 μg of folic acid is at least 600 μg of folic acid. In additional embodiments, the at least 20 mg of iron is at least 27 mg of iron. In further embodiments, the at least 125 mg of calcium is at least 900 mg of calcium.

In some embodiments, the tocopherol is present in the composition at at least 10 international units (IUs) (e.g., 10-40 IUs). In additional embodiments, the tocopherol is present in the composition at at least 31 international units (IUs) (e.g., 31-65 IU's). In further embodiments, the tocopherol is present in the composition at at least 51 international units (IUs) (e.g., 51 . . . 75 . . . 200 . . . or 500 IUs). In other embodiments, the tocopherol is present in the composition at at least 500 international units (IUs) (e.g., 500-750 IUs).

### DESCRIPTION OF THE FIGURES

FIGS. 1A-C shows α-Tocopherol (α-T)-supplemented diet inhibited eosinophil recruitment in pups from allergic mothers. FIG. 1A: schematic of the timeline for d-α-tocopherol diet and ovalbumin (OVA) treatment of mothers and pups. Mothers were sensitized and challenged with OVA or saline and then, at the time of breeding, they were given diets supplemented with d-α-tocopherol. The diets contained 150, 250, or 500 mg d-α-tocopherol/kg diet. On day 3 after birth, the pups were sensitized one time with OVA/potassium aluminum sulfate (alum) by ip injection and then challenged with aerosolized 3% OVA on days 10-12. FIGS. B and C: mice were treated as in A. On postnatal (PND) 13, bronchoalveolar lavage (BAL) neutrophils, eosinophils, monocytes, and lymphocytes were cytospun and counted by standard morphological criteria. n=12 Mice/group. *P<0.05 compared with the other groups.

FIG. 2 shows a flow cytometry scheme for pup lung analysis.

FIG. 3 shows a flow cytometry scheme for pup fetal liver analysis.

FIGS. 4A-C show maternal supplementation with d-α-tocopherol inhibited eosinophil inflammation in lung tissue of pups from allergic mothers. FIG. 4A: tissues were from mice in FIG. 1. Representative micrographs of perivascular regions in pup lung tissue stained with eosin and methyl green. Images were obtained with a ×20 objective on an Olympus Microscope. Arrows on the images indicate some of the eosin-labeled perivascular eosinophils. Also shown is an enlarged image of the pup lung that was from OVA-treated mothers with basal diet. FIG. 4B: mouse body weight. FIG. 4C: OVA-specific serum IgE as determined by ELISA. n=8-10 Mice/group.

FIGS. 5A-E show d-α-Tocopherol inhibited lung tissue cytokines and eotaxins in pups from allergic mothers. On PND13, the BAL and lung tissues were collected from mice in FIG. 1. FIGS. 5A-D: lung tissue was placed in RNAlater and then examined by quantitative PCR for IL-4, IL-33, eotaxin 1 (CCL11), and eotaxin 2 (CCL24). FIG. 5E: BAL supernatant was examined for CCL24 by ELISA (Raybiotech). n=8-10 Animals/group. *P<0.05 compared with the corresponding saline control.

FIGS. 6A-F show d-α-Tocopherol supplementation during pregnancy was sufficient for inhibition of allergic inflammation. FIG. 6A: schematic of the timeline for d-α-tocopherol diet and OVA treatment of mothers and pups. Mothers were sensitized and challenged with OVA or saline and then, at the time of breeding, they were given diets supplemented with d-α-tocopherol. The diets contained 250 mg d-α-tocopherol/kg diet. On the day of birth, the pups were cross-fostered as indicated. On PND3 after birth, the pups were sensitized one time with OVA/alum by ip injection and then challenged with aerosolized 3% OVA on PND10-12. FIG. 6B-E: mice were treated as in A. On PND13, BAL neutrophils, eosinophils, monocytes, and lymphocytes were cytospun and counted by standard morphological criteria. Saline basal αT with the right-pointing triangle and then OVA 0αT indicates the pups were born from mothers treated with saline and fed basal α-tocopherol diet and then after the arrow, it indicates the treatments of the mother that nursed the pup for the cross-foster. n=12 Mice/group. FIG. 6F: in another experiment with a set of mice separate from B-E, mice received basal diet and were treated as in A. On PND13, BAL neutrophils, eosinophils, monocytes, and lymphocytes were cytospun and counted by standard morphological criteria. Pups from allergic mothers cross-fostered to another allergic mother developed allergic responses as in pups from allergic mothers that were not cross-fostered. *P<0.05 compared with other groups. **P<0.05 compared with saline groups.

FIG. 7A-B show d-α-Tocopherol supplementation inhibited allergic inflammation in pups from allergic mothers that previously had basal diets and litters of allergic pups. FIG. 7A: schematic of timeline for 500 mg α-tocopherol/kg diet and OVA treatment of mothers and pups. B: saline or OVA-treated moms were on basal diet and had a litter of allergic pups (data in FIG. 1). Next, a group of these moms were switched at time of mating to 500 mg/kg diet for the second litter of pups. Lung lavage cells from pups were cytospun, and leukocytes were counted by standard morphological criteria. n=12 Mice/group. *P<0.05 compared with other groups for each cell type.

FIG. 8 shows expression of cytokines, chemokines, indolamine dioxygenase (IDO), and MHCII by lungs from pups. Lung tissue from the pups in FIG. 7 was collected 24 h after the last challenge, placed in RNAlater, and then examined by qPCR. **P<0.05 compared with all other groups. *P<0.05 compared with the saline/basal diet group.

FIGS. 9A-D show analysis of liver tocopherols and dendritic cells (DCs) in the pups and mothers. FIG. 9A: schematic of the timeline for d-α-tocopherol diet and OVA treatment of mothers and pups. On gestational day (GD) 18, tocopherols were determined in the liver of the mothers by HPLC, and the fetal liver was analyzed for dendritic cell subsets. With another set of mothers, on PND13, the pup livers were analyzed for tocopherol isoforms by HPLC, and the pup lungs were analyzed for BAL inflammation and lung tissue dendritic cell subsets. FIG. 9B: mother liver α-tocopherol. *P<0.05 compared with basal diet group. FIG. 9C: PND13 pup liver α-tocopherol. *P<0.05 compared with basal diet group. FIG. 9D: BAL eosinophils and monocytes were determined by cytospins and cell counting. *P<0.05 compared with other groups.

FIGS. 10 A-C show d-α-Tocopherol supplementation of allergic female mice reduced the numbers of CD11c+CD11b+DCs in the pup lung. The lung tissues were from the pups described in FIG. 9A. FIG. 10A: chart of lung CD11c+ subsets analyzed in the pup lungs. B: CD11c+CD11b+ subsets of DCs in the pup lung. The relative expression level (mean fluorescence intensity) for MHCII, CD80, and CD86 was not different among the groups (data not show). *P<0.05 compared with the saline, basal diet group or compared with the group indicated for alveolar DCs. **P<0.05 compared with pups from mothers with basal diets. FIG. 10C: CD11c+CD11b−dendritic cell subsets in the pup lung. *P<0.05 compared with groups with saline-treated mothers. mDC, monocyte-derived DCs; pDC, plasmacytoid DCs.

FIGS. 11A-C show d-α-Tocopherol supplementation of allergic female mice reduced the numbers of CD11c+CD11b+DCs in the fetal liver. The fetal liver tissues were from the pups described in FIG. 9A. FIG. 11A: chart of dendritic cell subsets detected in the GD18 fetal liver. B: CD11c+CD11b+mDCs, CD80+CD11c+CD11b+mDCs, and CD86+CD11c+CD11b+mDCs in the fetal liver and mean fluorescence intensity of CD80 and CD86 on these fetal liver DCs. *P<0.05 compared with groups with basal diet. FIG. 11C: CD11c+CD11b−resident DCs, CD80+CD11c+CD11b−resident DCs, and CD86+CD11c+CD11b−resident DCs in the fetal liver and mean fluorescence intensity of CD80 and CD86 on these fetal liver DCs. *P<0.05 compared with the other groups or compared with the groups indicated. MFI, mean fluorescence intensity.

FIGS. 12A-B show d-α-Tocopherol inhibited the generation of bone marrow-derived CD11c+CD11b+DCs in vitro. Bone marrow from PND10 pups was cultured for 10 days with GM-CSF in the presence of 0.01% DMSO (solvent control) or 80 μM d-α-tocopherol (as we previously described for in vitro cell loading with d-α-tocopherol) (7). FIG. 12A: no. of CD45+CD11c+CD11b+ cells. FIG. 12B: no. of cells with resident dendritic cell phenotype (CD45+CD11b+CD11c+Ly6c−MHCII−). There was no difference in the % live cells between the two groups. There were no cells from the culture with the monocyte-derived phenotype (CD45+CD11c+CD11b+Ly6c+, MHCII high) (data not shown). n=5-6 from a representative experiment of two experiments. *P<0.05 compared with the DMSO-treated control.

FIGS. 13A-B show that maternal γ-tocopherol-supplemented diet reduced the number of allergic mothers with pups. FIG. 13A shows the percentage of mated females that had pups, and FIG. 13B shows the number of pups per mom.

FIGS. 14A-B show maternal γ-tocopherol-supplemented diet elevates tissue γ-tocopherol in mothers (FIG. 14A) and pups (FIG. 14B).

FIGS. 15A-B show that a maternal γ-tocopherol-supplemented diet increases the number of BAL leukocytes and number of IRF4+CD11c+CD11b+ dendritic cells in OVA-challenged pups from allergic mothers. FIG. 15A shows the number of leukocytes in pup BAL, and FIG. 15B shows the number of IRFA+CD11b+ alveolar DCs per million pup lung cells.

FIGS. 16A-C show maternal γ-tocopherol diet increases inflammatory mediators in OVA-treated pup lungs. FIG. 16A shows the results from RayBiotech protein array (308 proteins) with 2 hour minced lung culture supernatants. Shown is fold change of protein from lungs of OVA-stimulated pups from γT-supplemented allergic mothers compared to lungs from allergic mothers fed basal diet. Above the red line is considered significant in the array. FIG. 16B shows an ELISA from culture supernants in panel A. FIG. 16C shows an ELISA for OVA-specific IgE in serum from pups.

FIGS. 17A-C show α-T and γ-T have opposing effects in regulation of DC development and function in vitro. FIGS. 17A and B show results from bone marrow from postnatal day 10 neonate (with basal diet) that was cultured with GM-CSF and with 80 μM αT (FIG. 17B) or 2 μM γT (FIG. 17A) for 8 days and analyzed for DC subsets by immunolabeling/flow cytometry. *, p<0.05 compared to DMSO group. **, p<0.05 compared to indicated group. FIG. 17 C shows results from bone marrow-derived DCs that were co-cultured 48 hours with purified CD4+ T cells with and without 80 μM α-T or 2 μM γ-T, or both tocopherols. Expression by qPCR. There was no effect on cell viability (not shown). n=3. **, p<0.05 compared to all other groups. *, p<0.05 compared to DMSO group.

### DETAILED DESCRIPTION

The present invention provides methods, compositions, and systems for preventing or reducing an allergic condition in an offspring by administering tocopherol to a mother pregnant or nursing the offspring, where the tocopherol in the composition is 98-100% unmodified natural d-alpha tocopherol, and less than 2% gamma tocopherol (e.g., undetectable levels of gamma tocopherol). In certain embodiments, a prenatal tablet or pill is provided composed of such tocopherol compositions in combination with folic acid, iron, and calcium.

Described in the Examples herein is the identification that supplementation of allergic female mice with α-tocopherol inhibited neonatal development of allergic responses. In addition, there was a reduction in neonatal cytokines, chemokines, and lung CD11b+ dendritic cell subsets, which are critical to development of allergic responses. α-Tocopherol also reduced CD11b+ dendritic cell subsets in the fetal liver and in vitro in bone marrow differentiated DCs. Therefore, in some embodiments, humans or other animal mothers are provided with alpha-tocopherol supplements (with un-detectable or almost undetectable levels of gamma-tocopherol) before becoming pregnant, during pregnancy, and/or during any breast feeding, such that offspring have reduced levels of allergies (and other inflammation).

It is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The formulas are shown without a definitive stereochemistry at certain positions. The present invention includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

It must also be noted that as used herein and in the appended claims, the singular forms “a”, “an”, and “the” include plural reference unless the context clearly dictates otherwise. Thus, for example, reference to a “fibroblast” is a reference to one or more fibroblasts and equivalents thereof known to those skilled in the art, and so forth.

As used herein, the term “about” means plus or minus 10% of the numerical value of the number with which it is being used. Therefore, about 50% means in the range of 45%-55%.

By “pharmaceutically acceptable”, it is meant the carrier, diluent or excipient must be compatible with the other ingredients of the formulation and not deleterious to the recipient thereof.

A “therapeutically effective amount” or “effective amount” of a composition is a predetermined amount calculated to achieve the desired effect. The activity contemplated by the present methods includes both medical therapeutic and/or prophylactic treatment, as appropriate (e.g., administration of alpha-tocopherol to mothers to reduce allergies in offspring). The specific dose of a compound administered according to this invention to obtain therapeutic and/or prophylactic effects will, of course, be determined by the particular circumstances surrounding the case, including, for example, the compound administered, the route of administration, and the condition being treated. The compounds are effective over a wide dosage range and, for example, dosages per day will normally fall within the range of from 0.001 to 10 mg/kg, more usually in the range of from 0.01 to 1 mg/kg. However, it will be understood that the effective amount administered will be determined by the physician in the light of the relevant circumstances including the condition to be treated, the choice of compound to be administered, and the chosen route of administration, and therefore the above dosage ranges are not intended to limit the scope of the invention in any way. A therapeutically effective amount of compound of this invention is typically an amount such that when it is administered in a physiologically tolerable excipient composition, it is sufficient to achieve an effective systemic concentration or local concentration in the tissue.

The terms “treat,” “treated,” or “treating” as used herein refers to both therapeutic treatment and prophylactic or preventative measures, wherein the object is to prevent or slow down (lessen) an undesired physiological condition, disorder or disease, or to obtain beneficial or desired clinical results. For the purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment.

In some embodiments, the compounds and methods disclosed herein can be utilized with or on a subject in need of such treatment, which can also be referred to as “in need thereof.” As used herein, the phrase “in need thereof” means that the subject has been identified as having a need for the particular method or treatment and that the treatment has been given to the subject for that particular purpose.

Some embodiments described herein are compositions comprising a tocopherol, tocotrienol or a combination thereof. In some embodiments, the composition consists essentially of a tocopherol. In some embodiments, the composition consists of α-tocopherol. Some embodiments are directed to a composition comprising at least one tocopherol, tocotrienol or a combination thereof. In some embodiments, the tocopherol is α-tocopherol. In some embodiments, the α-tocopherol comprises at least about 90%, at least about 95%, at least about 98%, at least about 99%, at least about 99.5%, or at least about 99.95% of the composition. In some embodiments the α-tocopherol comprises about 100% of the composition. In certain embodiments, un-detectable or nearly un-detectable levels of gamma-tocopherol are present with the alpha-tocopherol (e.g., of all the tocopherol in a composition, at least 99.9% is alpha, and 0.1% or less is gamma tocopherol).

Some embodiments described are pharmaceutical compositions comprising a tocopherol, tocotrienol or a combination thereof. In some embodiments, the tocopherol is α-tocopherol. In some embodiments, the pharmaceutical composition consists essentially of alpha tocopherol. In some embodiments, the pharmaceutical composition consists essentially of α-tocopherol. Some embodiments are directed to a pharmaceutical composition comprising at least one tocopherol, tocotrienol or a combination thereof. In some embodiments, the tocopherol is α-tocopherol. In some embodiments, the α-tocopherol comprises at least about 90%, at least about 95%, at least about 98%, at least about 99%, at least about 99.5%, or at least about 99.95% of the pharmaceutical composition. In some embodiments the α-tocopherol comprises about 100% of the pharmaceutical composition.

In some embodiments, the tocopherol, tocotrienol or a combination thereof is present in a therapeutically effective amount. Some embodiments are directed to a method of treating a condition in a patient in need thereof comprising administering a pharmaceutical composition to said patient (e.g., pregnant mother, nursing mother, or woman attempting to become pregnant).

In some embodiments, the patient is a human. In some embodiments, the patient is selected from the group consisting of an unborn infant, neonate, an infant, and a child. In some embodiments, the patient is not an adult. In some embodiments, an adult is defined as a human being that is of reproductive age.

In some embodiments, the condition is premature fetal lung disease, full term pulmonary distress, childhood asthma or a combination thereof. In other embodiments, the condition is an allergic condition. In some embodiments, the condition is an allergic condition associated with the respiratory system.

Some embodiments are directed to a method of preventing a condition in an neonate, infant or child comprising administering a pharmaceutical composition to the mother of the neonate, infant or child prior to birth of the neonate, infant or child. In some embodiments, the pharmaceutical compositions comprise a tocopherol, tocotrienol or a combination thereof. In some embodiments, the tocopherol is α-tocopherol. In some embodiments, the pharmaceutical composition consists essentially of a tocopherol. In some embodiments, the pharmaceutical composition consists essentially of α-tocopherol. In some embodiments, the tocopherol, tocotrienol or a combination thereof is present in a therapeutically effective amount. In some embodiments, the condition is premature fetal lung disease, full term pulmonary distress, childhood asthma or a combination thereof. In other embodiments, the condition is an allergic condition. In some embodiments, the condition is an allergic condition associated with the respiratory system.

In some embodiments, the tocopherol, tocotrienol or a combination thereof is present in a therapeutically effective amount. In some embodiments, the patient is a human. In some embodiments, the patient is selected from the group consisting of a neonate, an infant, and a child. In some embodiments, the patient is not an adult. In some embodiments, an adult is defined as a human being that is of reproductive age.

In some embodiments, the condition is premature fetal lung disease, full term pulmonary distress, childhood asthma or a combination thereof. In other embodiments, the condition is an allergic condition. In some embodiments, the condition is an allergic condition associated with the respiratory system.

Some embodiments are directed to a method of preventing a condition in a neonate, infant or child comprising administering a composition to the mother of the neonate, infant or child prior to birth of the neonate, infant or child. In some embodiments, the compositions comprise a tocopherol, tocotrienol or a combination thereof. In some embodiments, the tocopherol is α-tocopherol, β-tocopherol, γ-tocopherol, δ-tocopherol or a combination thereof. In some embodiments, the tocotrienol is a d-α, d-β, d-γ, or d-δ-tocotrienol. In some embodiments, the composition consists essentially of a tocopherol. In some embodiments, the composition consists essentially of α-tocopherol.

In some embodiments, administration of the compositions described herein is to a pregnant subject (i.e., a human female) during the course of pregnancy may reduce the incidence of certain conditions in the subject's neonate, infant or child. In some embodiments, the condition is premature fetal lung disease, full term pulmonary distress, childhood asthma or a combination thereof. In other embodiments, the condition is an allergic condition. In some embodiments, the condition is an allergic condition associated with the respiratory system.

In some embodiments, administration of the compositions described herein is directly to the infant in utero. In some embodiments, the compositions can be administered to an unborn infant indirectly via the mother. In some embodiments, administration of the compositions to the subject continues after birth of the infant. In some embodiments, the compositions may be administered to the neonate, infant or child via the subject's breast milk. In some embodiments, the compositions may be administered to the subject for as long as the subject is breast-feeding the neonate, infant or child. In some embodiments, the compositions may be administered to the subject for a period of about 1 week about 2 weeks, about 3 weeks, about 1 month, about 2-3 months, about 3-6 months, about 6-9 months, about 12-18 months, about 18 to 24 months or about 24 to 30 months.

In some embodiments, the compositions are administered directly to the neonate, infant, or child after birth. In some embodiments, the compositions may be administered to the neonate, infant, or child for a period of about 1 week about 2 weeks, about 3 weeks, about 1 month, about 2-3 months, about 3-6 months, about 6-9 months, about 12-18 months, about 18 to 24 months or about 24 to 30 months after birth.

For example, in some aspects, the invention is directed to a pharmaceutical composition comprising a compound, as defined above, and a pharmaceutically acceptable carrier or diluent, or an effective amount of a pharmaceutical composition comprising a compound as defined above. The compounds of the present invention can be administered in the conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the present invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Specific modes of administration will depend on the indication. The selection of the specific route of administration and the dose regimen is to be adjusted or titrated by the clinician according to methods known to the clinician in order to obtain the optimal clinical response. The amount of compound to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

Pharmaceutical formulations containing the compounds of the present invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a polymer or copolymer of the present invention. It is also known in the art that the active ingredients can be contained in such formulations with pharmaceutically acceptable diluents, fillers, disintegrants, binders, lubricants, surfactants, hydrophobic vehicles, water-soluble vehicles, emulsifiers, buffers, humectants, moisturizers, solubilizers, preservatives and the like. The means and methods for administration are known in the art and an artisan can refer to various pharmacologic references for guidance. For example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980) can be consulted.

The compounds of the present invention can be formulated for parenteral administration by injection, e.g., by bolus injection or continuous infusion. The compounds can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as, e.g., lactose, binders such as, e.g., starches, and/or lubricants such as, e.g., talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of, e.g., tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch. The compounds of the present invention can also be formulated in rectal compositions such as suppositories or retention enemas, e.g., containing conventional suppository bases such as cocoa butter or other glycerides.

In addition to the formulations described previously, the compounds of the present invention can also be formulated as a depot preparation. Such long acting formulations can be administered by implantation (for example subcutaneously or intramuscularly) or by intramuscular injection.

Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the present invention, for example, can be applied to a plaster, or can be applied by transdermal, therapeutic systems that are consequently supplied to the organism. Pharmaceutical compositions of the compounds also can comprise suitable solid or gel phase carriers or excipients. Examples of such carriers or excipients include but are not limited to calcium carbonate, calcium phosphate, various sugars, starches, cellulose derivatives, gelatin, and polymers such as, e.g., polyethylene glycols.

The compounds of the present invention can also be administered in combination with other active ingredients, such as, for example, adjuvants, protease inhibitors, or other compatible drugs or compounds where such combination is seen to be desirable or advantageous in achieving the desired effects of the methods described herein.

In some embodiments, the disintegrant component comprises one or more of croscarmellose sodium, carmellose calcium, crospovidone, alginic acid, sodium alginate, potassium alginate, calcium alginate, an ion exchange resin, an effervescent system based on food acids and an alkaline carbonate component, clay, talc, starch, pregelatinized starch, sodium starch glycolate, cellulose floc, carboxymethylcellulose, hydroxypropylcellulose, calcium silicate, a metal carbonate, sodium bicarbonate, calcium citrate, or calcium phosphate.

In some embodiments, the diluent component comprises one or more of mannitol, lactose, sucrose, maltodextrin, sorbitol, xylitol, powdered cellulose, microcrystalline cellulose, carboxymethylcellulose, carboxyethylcellulose, methylcellulose, ethylcellulose, hydroxyethylcellulose, methylhydroxyethylcellulose, starch, sodium starch glycolate, pregelatinized starch, a calcium phosphate, a metal carbonate, a metal oxide, or a metal aluminosilicate.

In some embodiments, the optional lubricant component, when present, comprises one or more of stearic acid, metallic stearate, sodium stearyl fumarate, fatty acid, fatty alcohol, fatty acid ester, glyceryl behenate, mineral oil, vegetable oil, paraffin, leucine, silica, silicic acid, talc, propylene glycol fatty acid ester, polyethoxylated castor oil, polyethylene glycol, polypropylene glycol, polyalkylene glycol, polyoxyethylene-glycerol fatty ester, polyoxyethylene fatty alcohol ether, polyethoxylated sterol, polyethoxylated castor oil, polyethoxylated vegetable oil, or sodium chloride.

### EXAMPLES

### Example 1

### Supplementation of Allergic Female Mice with α-Tocopherol Inhibits Neonatal Development of Allergic Responses

This example describes that supplementation of allergic female mice with α-tocopherol inhibited neonatal development of allergic responses. Allergic female mice were supplemented with α-tocopherol starting at mating. The pups from allergic mothers developed allergic lung responses, whereas pups from saline-treated mothers did not respond to the allergen challenge and α-tocopherol supplementation of allergic female mice resulted in a dose-dependent reduction in eosinophils in the pup bronchoalveolar lavage and lungs after allergen challenge. There was also a reduction in pup lung CD11b⁺ dendritic cell subsets that are critical to development of allergic responses, but there was no change in several CD11b⁻ dendritic cell subsets. Furthermore, maternal supplementation with α-tocopherol reduced the number of fetal liver CD11b⁺ dendritic cells in utero. In the pups, there was reduced allergen-induced lung mRNA expression of IL-4, IL-33, TSLP, CCL11, and CCL24. Cross-fostering pups at the time of birth demonstrated that α-tocopherol had a regulatory function in utero. In conclusion, maternal supplementation with α-tocopherol reduced fetal development of subsets of dendritic cells that are critical for allergic responses and reduced development of allergic responses in pups from allergic mothers.

### Materials and Methods

### Animals

C57BL/6 mice were from Jackson Laboratories (Bar Harbor, Me.). The studies are approved by the Northwestern University Institutional Review Committee for animals.

### Tocopherol Reagents

D-α-Tocopherol (>98% pure) from Sigma was sent to Dyets (Bethlehem, Pa.) to make the diets with 150 mg α-tocopherol/kg diet (catalog no. 103372), 250 mg α-tocopherol/kg diet (catalog no. 103373), and 500 mg α-tocopherol/kg diet (catalog no. 103296) (Table 1). The purity of these tocopherols that were used to make the diets and the tocopherol concentrations in the diets was confirmed by HPLC with electrochemical detection as described below.

TABLE 1
α-Tocopherol-supplemented diets (modified AIN-93G
Purified Rodent Diet with corn oil replacing soybean oil)

| | g/kg diet |
|---|---|
| Casein, high nitrogen | 200 |
| L-Cystine | 3 |
| Sucrose | 100 |
| Cornstarch | 397 |
| Dyetrose | 132 |
| Corn oil | 70 |
| Cellulose | 50 |
| Mineral mix no. 210025 | 35 |
| Vitamin mix no. 210025 | 10 |
| Choline bitartrate | 2.5 |
| D-α-tocopherol | 0.5, 0.25, or 0.15 |

The α-tocopherol-supplemented diet (500 mg/kg diet no. 103296, 250 mg/kg diet no. 103373, or 150 mg/kg diet no. 103372) and the basal control diet without supplementation (45 mg α-tocopherol/kg diet) (diet no. 101591) were from Dyets. Corn oil, which was commonly used in rodent diet in the past, was used in this diet instead of the more recent formulas with soybean oil to avoid the proinflammatory contribution of high γ-tocopherol in soybean oil (5, 17, 44).

### Ovalbumin/Tocopherol Administration and Inflammation

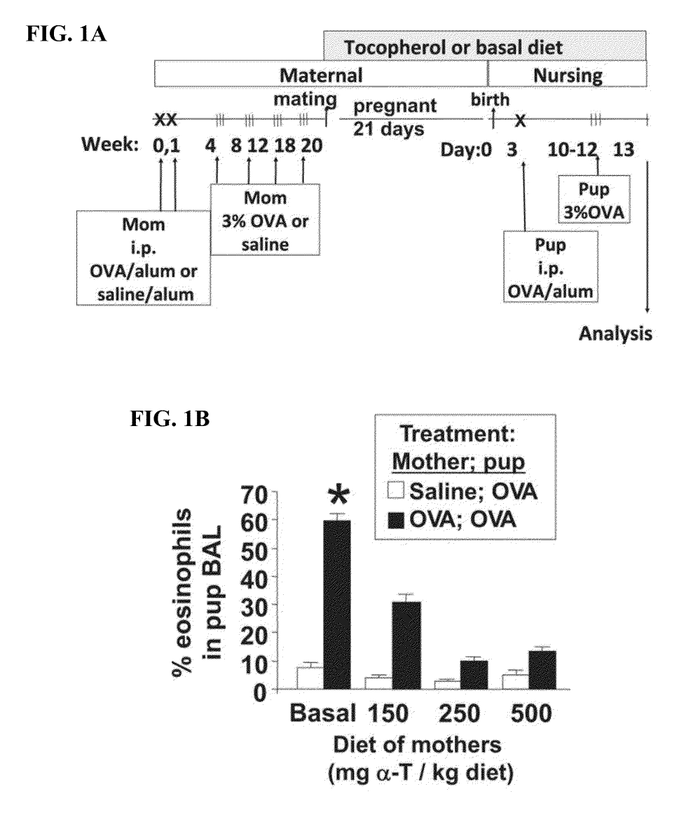

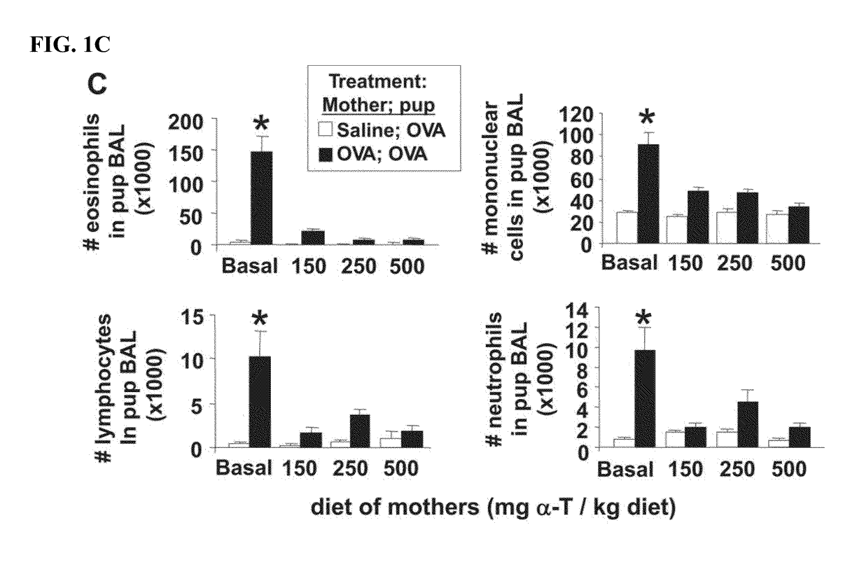

C57BL/6 female mice were maintained on chow diet. The mice were sensitized by intraperitoneal injection (200 μl) of ovalbumin (OVA) grade V (5 μg)/potassium aluminum sulfate (alum, 1 mg) or saline/alum (1 mg) on days 0 and 7 (26, 28, 46). The mice were exposed to nebulized saline or 3% (wt/vol) OVA in saline for 15 min on three consecutive days at 8, 12, and 16 wk of age. At 18 wk old, the female mice were challenged one time with 3% OVA (26, 28, 46) and then fed basal diet or diet supplemented with D-α-tocopherol (500 mg α-tocopherol/kg diet) (Table 1) (42, 52, 71). Basal, rather than deficient, tocopherol diets are used because α-tocopherol is necessary for placental development (37, 54). The 250 to 500 mg D-α-tocopherol/kg diet doses were chosen for these studies because it is commonly used to elevate tissue tocopherols and regulate immune responses in adult rodents (52, 53, 69), and it is 30 times lower than the high maternal tocopherol diet dose reported to reduce hippocampus function in rodents (8). Ten days after starting the tocopherol-supplemented diet, females were mated with normal males. Three-day-old pups were sub-optimally sensitized by treating with only one 50-μl ip injection (rather than two injections) of 5 μg OVA/1 mg alum (26, 28, 33). At 10, 11, and 12 days old, the pups were challenged for 15 min with 3% OVA (FIG. 1A). At 13 days old, the pups were weighed and killed, and tissues were collected. Serum was collected for analysis of IgE, liver was for tocopherol analysis, lung lavage was for leukocytes and cytokines, and lung tissue was for histology, flow cytometry, and qPCR of mediators of allergic inflammation. Liver was extracted and examined for tocopherols by high-pressure liquid chromatography with electrochemical detection as we previously described (7, 50). Bronchoalveolar lavage (BAL) cells were stained and counted as previously described (3). OVA-specific IgE was determined by ELISA as previously described (13). Lung tissue sections were fixed in cold methanol for 15 min, rehydrated for 60 min with PBS, and stained with eosin and methyl green as we previously described (6).

### Tocopherol Measurement

Diets or livers were weighed and homogenized in absolute ethanol with 5% ascorbic acid on ice. The internal standard tocol is added to each lung to determine recovery. The homogenate or plasma was extracted with an equal volume of hexane with 0.37 wt/100 wt butylated hydroxytoluene to prevent oxidation and increase recovery of tocopherol. The samples were vortexed and then centrifuged for 10 min at 9,000 g at 4° C. The hexane layer was dried under nitrogen and stored at −20° C. The samples were reconstituted in methanol, and then tocopherols were separated using a reverse-phase C18 HPLC column (Hewlett Packard) and HPLC (Waters) with 99% methanol-1% water as a mobile phase with detection with an electrochemical detector (potential 0.7 V) (Waters).

### Cytokines, Chemokines, and Indolamine Dioxygenase

Total RNA was isolated from 50 to 100 mg lung tissue using the QIAGEN RNeasy Fibrous Tissue Mini Kit. cDNA was prepared using a SuperScript II RNase H-Reverse Transcriptase kit (Invitrogen) and analyzed by PCR on an ABI 7300 Thermal Cycler (Applied Biosystems). Taqman probes and Taqman Universal Master Mix were used as directed (Applied Biosystems). CCL24 in the BAL also was examined by ELISA (Raybiotech).

### Flow Cytometry Analysis of Pup Lung, Fetal Liver, and Cultured DCs

Following BAL of pups, lungs were removed for analysis of dendritic cell types by flow cytometry. In the fetus, hematopoiesis occurs in the fetal livers. Gestational day 18 fetal livers were collected for analysis of fetal liver DCs. Briefly, tissues were minced and suspended in 5 ml of RPMI solution containing 1 mg/ml collagenase D (Roche) and 0.2 mg/ml DNase I (Roche) at 37° C. with agitation. After 1 h, samples were filtered through sterile 70-μm mesh tissue, centrifuged, and resuspended in 5% FBS-RPMI solution. Red blood cells were lysed in 1×BD PharmLyse Lysing Buffer (BD Biosciences), and the cells were washed two times in PBS. All centrifugation steps were carried out at 300 g for 5 min. Three million cells were used per sample for immunolabeling.

For bone marrow-derived postnatal day 10 pup dendritic cell cultures, a single cell suspension of bone marrow from 10-day-old mice was placed in RPMI 1640, glutamine, HEPES, gentamycin sulfate, penicillin/streptomycin, and 10% heat-inactivated FCS. The bone marrow cells were treated with the DMSO solvent control (0.01%) or D-α-tocopherol (80 μM) as we previously described for in vitro treatment with D-α-tocopherol (7). The cells were stimulated with 5 ng granulocyte macrophage colony-stimulating factor (GM-CSF) per milliliter. The culture medium with tocopherol and GM-CSF was replaced on days 2, 4, and 7. On day 9, the cells were counted, immunolabeled with the markers described above for DCs, and examined by flow cytometry.

The cells were stained for live/dead exclusion in 500 μl PBS containing 0.25 μl of Aqua fluorescent reactive dye (Molecular Probes, Invitrogen) for 20 min at room temperature in the dark. Next, FC receptors were blocked by incubating the cells in 50 μl flow cytometry staining buffer (Biolegend) with 0.75 μl purified rat anti-mouse CD16/CD32 Mouse BD FC Block (no. 553142; BD Pharmingen) for 10 min at 4° C.

To prepare the antibodies for immunolabeling, for each sample equivalent, an antibody stock was prepared by adding the following antibodies to 50 μl of flow cytometry buffer: 1) Live/Dead Aqua fluorescent dye (no. L34957; Molecular Probes, Invitrogen), 0.25 μl/sample; 2) CD45, rat anti-mouse FITC-conjugated, 0.5 mg/ml, clone 30-F11 (no. 103107; Biolegend), 0.31 μl/sample; 3) CD11b, rat anti-mouse PE-CF594-conjugated, 0.2 mg/ml, clone M1/70 (no. 562317; BD Biosciences), 0.05 μl/sample; 4) Ly-6C, rat anti-mouse APC/Cy7-conjugated, 0.2 mg/ml, clone HK1.4 (no. 128025; Biolegend), 0.4 μl/sample; 5) CD11c, Armenian hamster anti-mouse PE/Cy7-conjugated, 0.2 mg/ml, clone N418 (no. 117317; Biolegend), 0.2 μl/sample; 6) I-A/I-E (MHCII), rat anti-mouse PerCP/Cy5.5-conjugated, 0.2 mg/ml, clone M5/114.15.2 (no. 107625; Biolegend), 0.1 μl/sample; 7) CD103, Armenian hamster anti-mouse Brilliant Violet 421-conjugated, clone 2E7 (no. 121421; Biolegend), 0.2 μl/sample; 8) CD317 (PDCA-1), rat anti-mouse APC-conjugated, 0.2 mg/ml, clone 927 (no. 127015; Biolegend), 0.5 μl/sample; 9) CD80, Armenian hamster anti-mouse PE-conjugated, 0.2 mg/ml, clone 16-10A1 (no. 104707; Biolegend), 0.5 μl/sample; and 10) CD86, rat anti-mouse Alexa Fluor 700-conjugated, 0.5 mg/ml, clone GL-1 (no. 105023; Biolegend), 0.5 μl/sample. Next, 50 μl of the antibody stock were added to the samples that were treated with FC block. The samples were incubated for 30 min at 4° C. in the dark, and the cells were washed two times in 1×PBS.

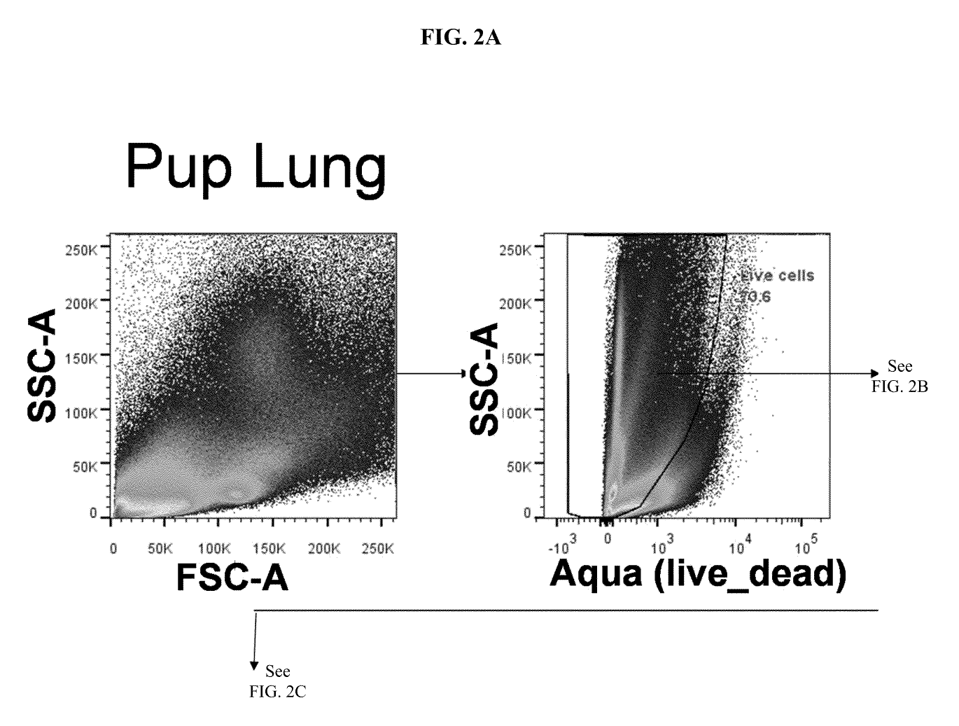

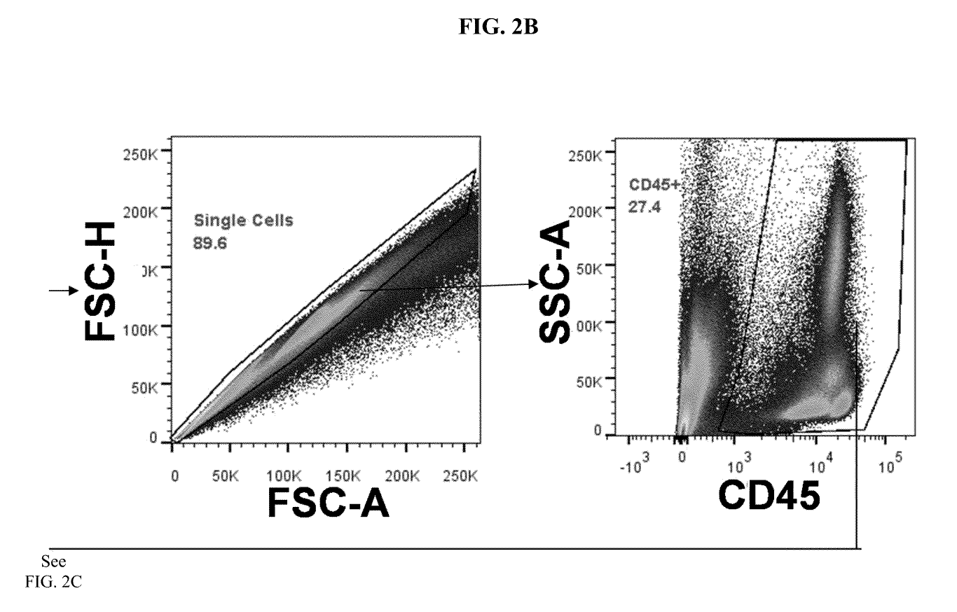

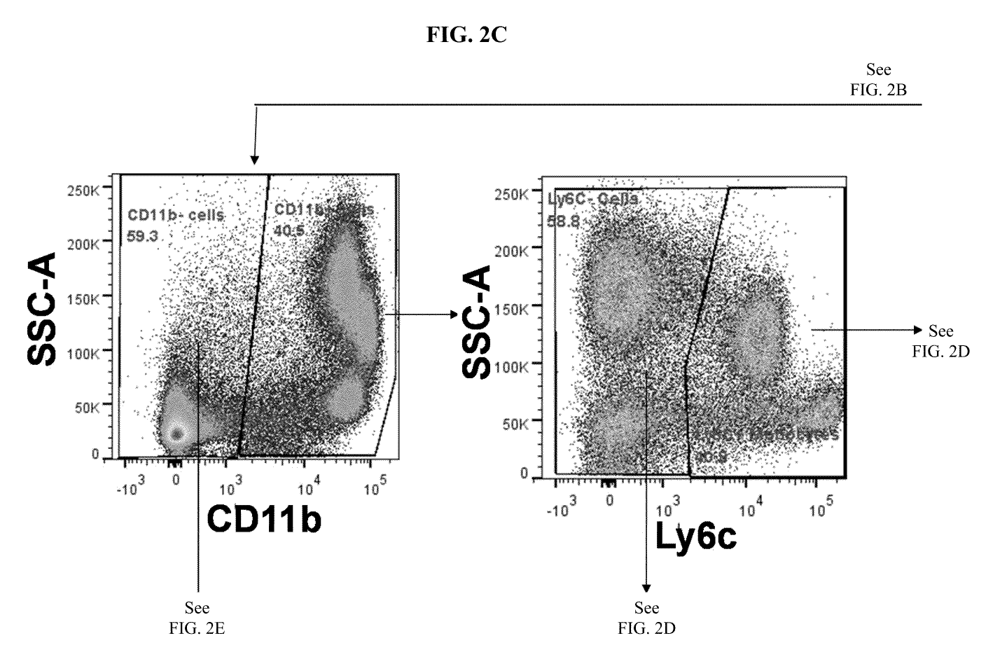

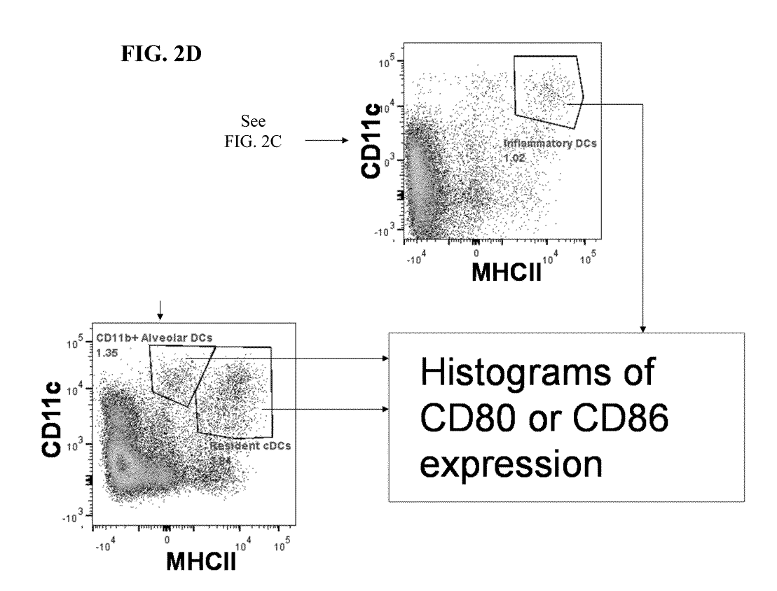

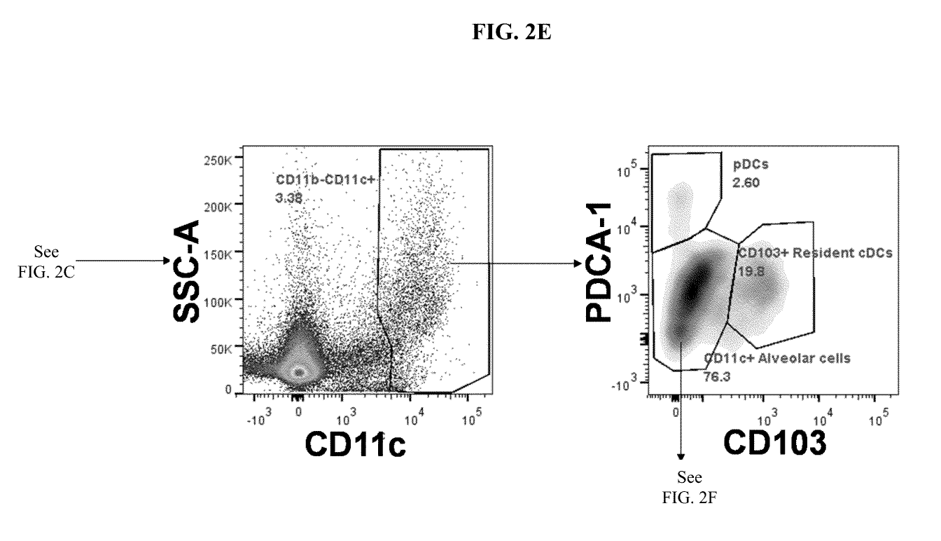

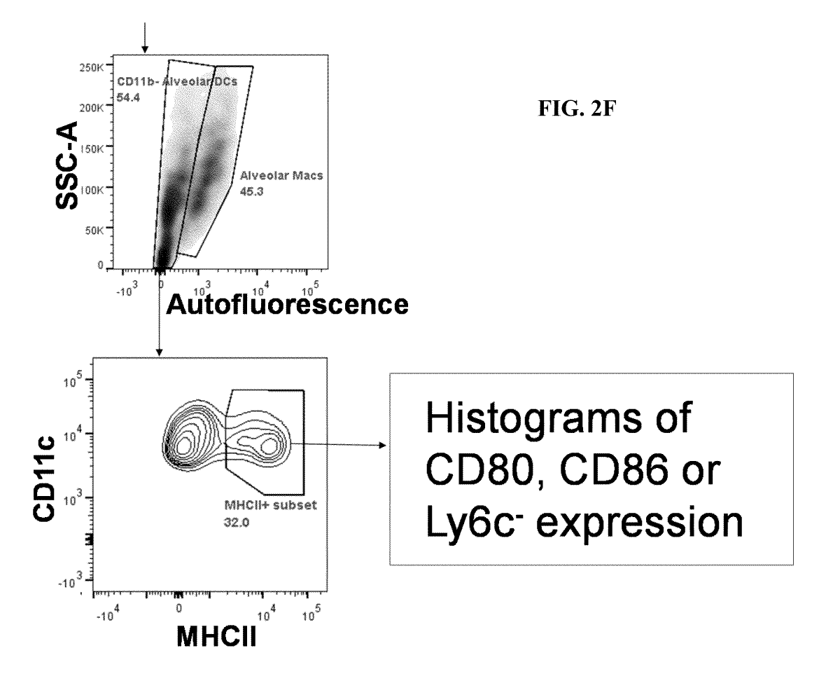

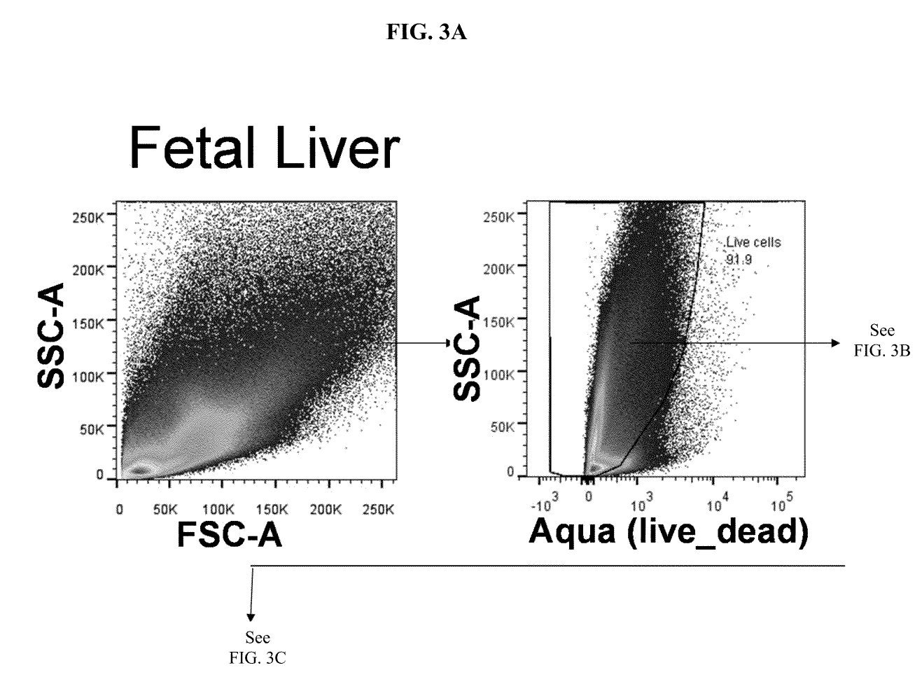

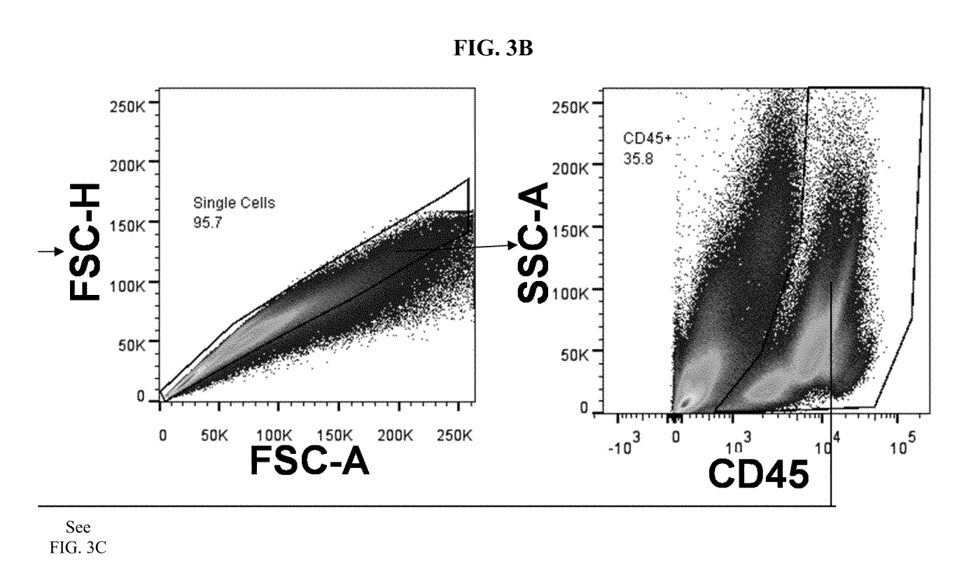

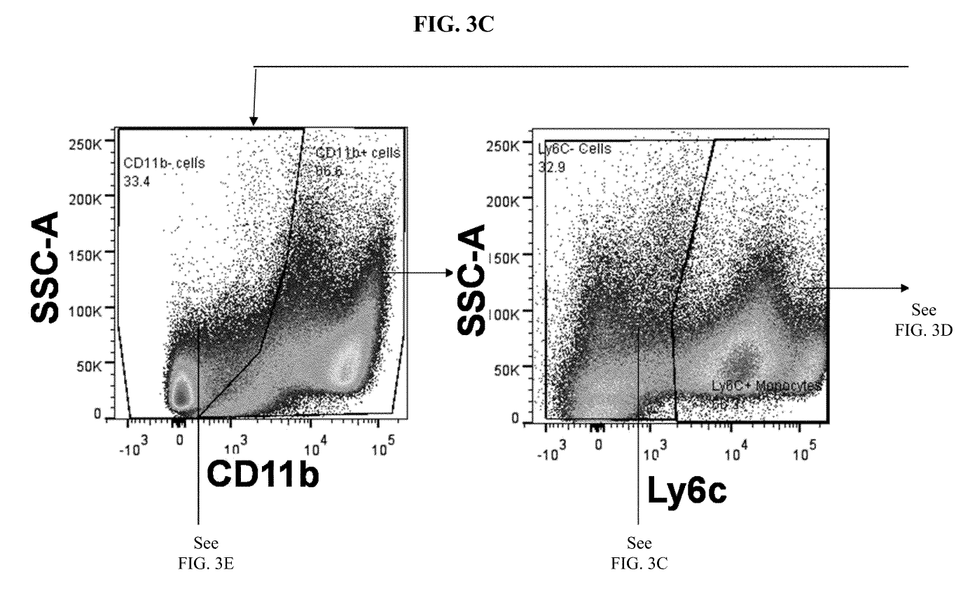

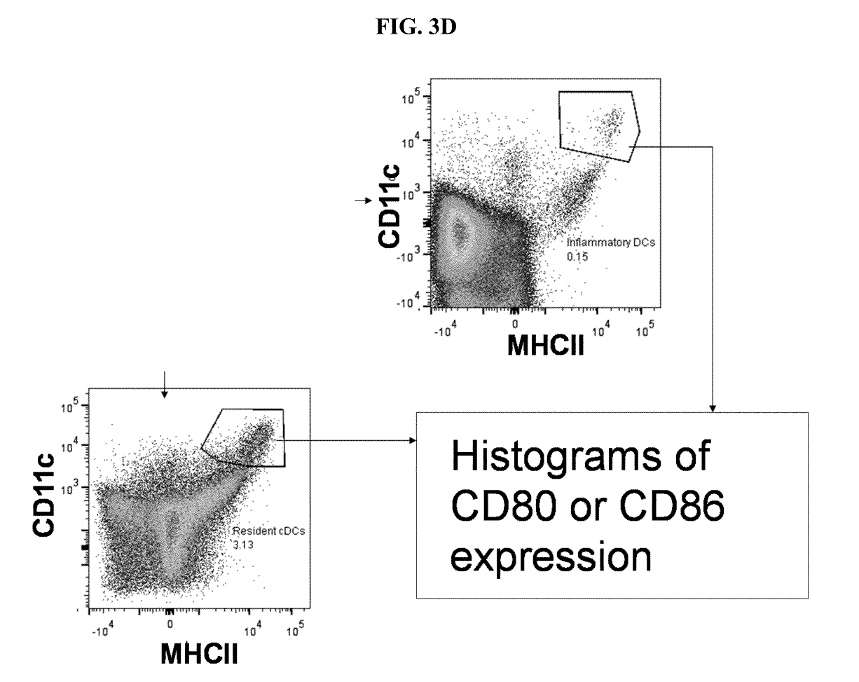

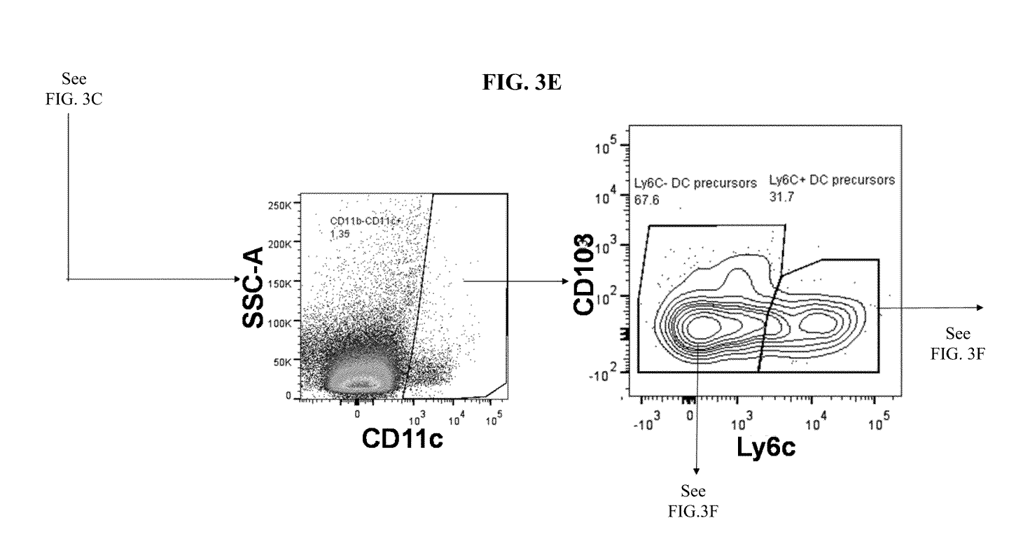

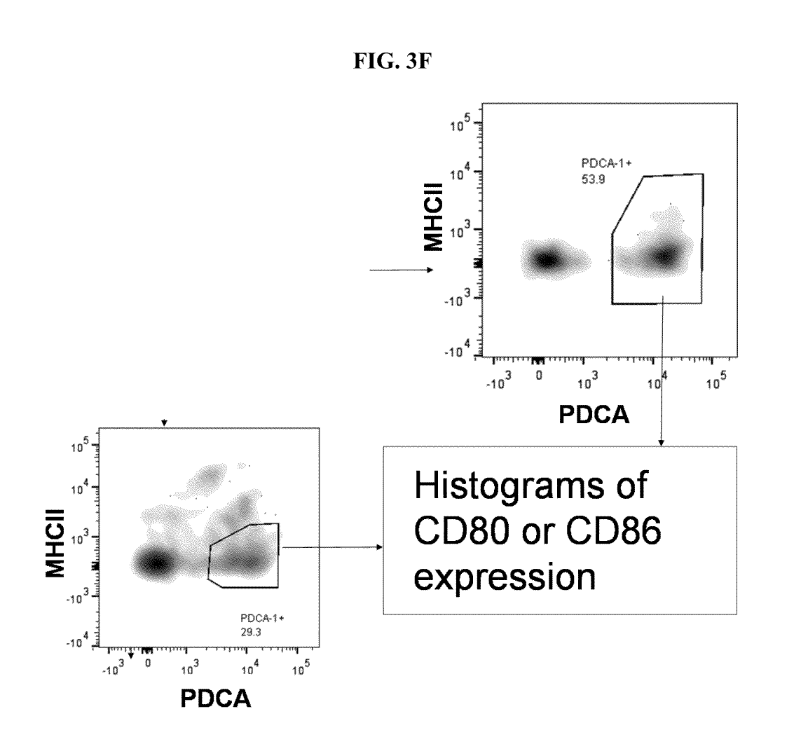

The cells were examined with a BD LSRII flow cytometer (BD Biosciences). Analysis was performed using FlowJo VX software (TreeStar). Compensation was done using FlowJo compensation wizard based on single color control staining of compensation beads (eBioscience). Nonstained controls were used to assess boundaries of live and dead populations. Only live, singlet (based on FSC-H vs. FSC-A gating), hematopoietic (CD45+) cells were used for subsequent gating of all populations. Fluorescence Minus One staining controls were used as negative controls to identify gates for populations of interest. The following subpopulations of DCs were analyzed: 1) resident conventional DCs: CD11b+Ly6C−CD11chighMHCIIhigh; 2) CD11b+ alveolar DCs: CD11b+Ly6C−CD11c+MHCII−; 3) inflammatory DCs: CD11b+Ly6C+CD11c+MHCII+; 4) plasmacytoid DCs (pDCs): CD11b−Ly6C−CD11clowPDCA-1+MHCII−; 5) resident CD103+DCs: CD11b−Ly6C−CD11c+CD103+MHCII−; and 6) CD11b−alveolar DCs: CD11b−Ly6C−CD11c+MHCII+/−. The flow cytometry gating strategy is in FIGS. 2 and 3.

### Statistics

Data were analyzed by a one-way ANOVA followed by Tukey's or Dunnett's multiple-comparisons test (SigmaStat; Jandel Scientific, San Ramon, Calif.). Presented are the means±SE.

### Results

### D-α-Tocopherol Supplementation of Allergic Female Mice Inhibits Allergic Inflammation in Neonates

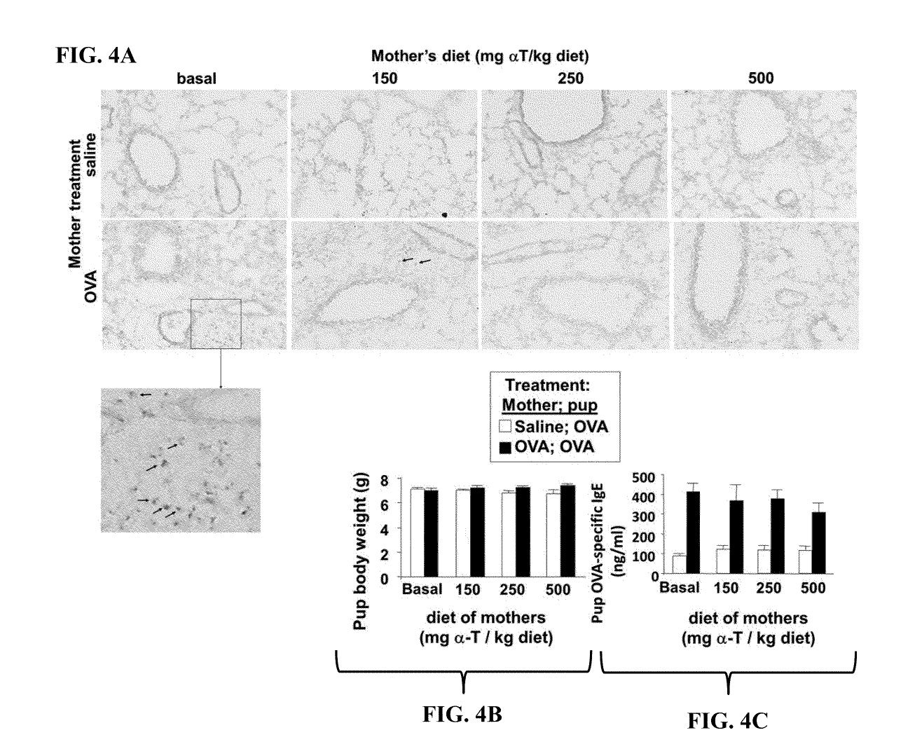

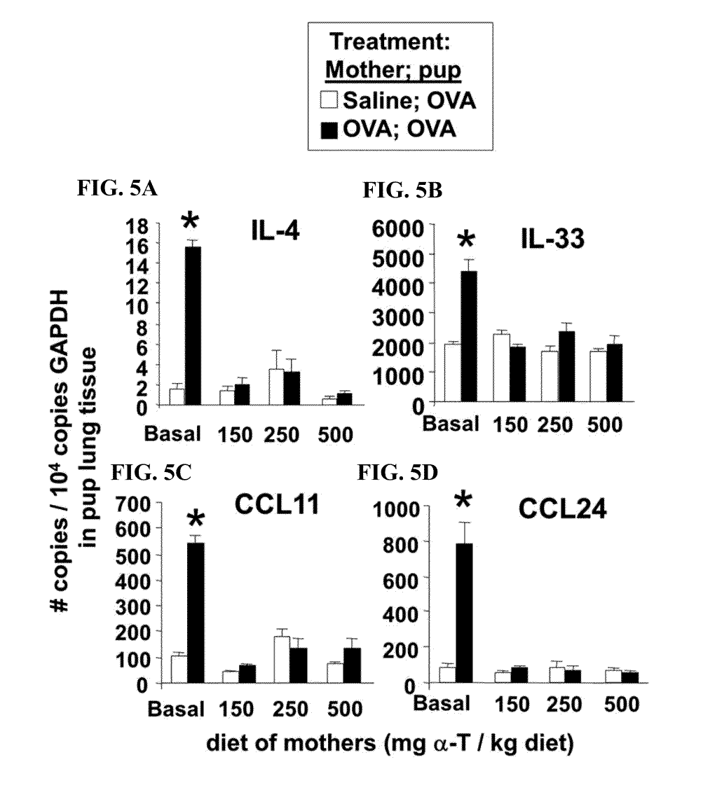

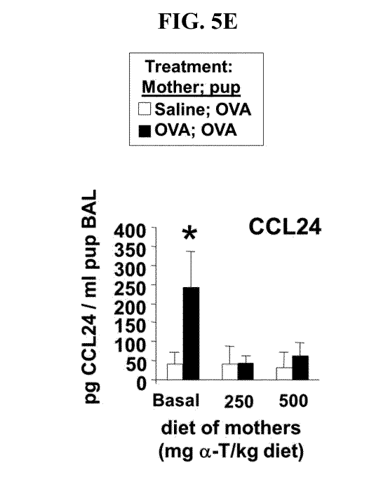

It was determined whether D-α-tocopherol supplementation of mothers during pregnancy/lactation inhibits development of allergic inflammation in their pups. In these studies, allergic responses were induced in 6-wk-old adult female mice by sensitization with OVA/alum at weeks 0 and 1 and challenge with OVA or saline three times a week during weeks 4, 8, 12, and 18 as in the timeline in FIG. 1A. Next, the females were supplemented with D-α-tocopherol at the time of mating (FIG. 1A). The mothers were given a standard basal diet (45 mg D-α-tocopherol/kg diet) or supplemented with 150, 250, or 500 mg D-α-tocopherol/kg diets (Table 1). A basal D-α-tocopherol diet was used as the control diet instead of a D-α-tocopherol-deficient diet because D-α-tocopherol is required for placenta development (37, 54). All pups from the mothers were challenged with a suboptimal regimen of OVA as previously described (46); the pups received one, instead of two, sensitizations with OVA-alum and then received three challenges with aerosolized OVA (FIG. 1A). The pups received tocopherol in utero and during lactation because it is reported that there is a contribution in utero and during nursing for the development of allergic responses in the offspring of allergic mothers (46). Compared with OVA-challenged pups from nonallergic saline-treated mothers, the OVA-treated pups from allergic mothers with basal diet had a significant increase in percent eosinophils in the BAL (FIG. 1B), an increase in total number of BAL eosinophils and monocytes (FIG. 1C), and an increase in the low numbers of BAL lymphocytes and neutrophils (FIG. 1C); this is consistent with previous reports (46). In contrast, α-tocopherol supplementation of the allergic mothers resulted in a dose-dependent inhibition of allergic inflammation in the OVA-stimulated pups from allergic mothers (FIGS. 1, B and C). The 150 mg α-tocopherol/kg diet partially inhibited the percent eosinophils in the BAL, whereas the 250 and 500 mg α-tocopherol/kg diet had the greatest inhibition of the percent eosinophils in the BAL (FIG. 1B). Therefore, all subsequent studies were performed using the 250 or 500 mg α-tocopherol/kg diets. α-Tocopherol supplementation of allergic female mice also reduced eosinophils in the lung tissue of OVA-challenged pups (FIG. 4A). There was no effect of treatments on pup weight (FIG. 4B), pup numbers, or pup gender distribution (data not shown). OVA-treated pups from allergic mothers had increased serum IgE, but serum IgE was not different among the D-α-tocopherol treatment groups (FIG. 4C). Because inflammation is regulated by Th2 cytokines and chemokines, we analyzed expression of several cytokines and chemokines involved in allergic inflammation. D-α-Tocopherol supplementation of allergic female mothers inhibited OVA-induced pup lung mRNA expression of IL-4, IL-33, CCL11, and CCL24 (FIG. 5, A-D) and protein expression of CCL24 (FIG. 5E). Therefore, D-α-tocopherol supplementation of allergic mothers inhibited allergic inflammation and cytokine/chemokine mediators of allergic inflammation in OVA-challenged pups from these allergic mothers.

### D-α-Tocopherol Supplementation of the Allergic Mothers During Pregnancy was Sufficient for Inhibition of Allergic Inflammation in Cross-Fostered Offspring

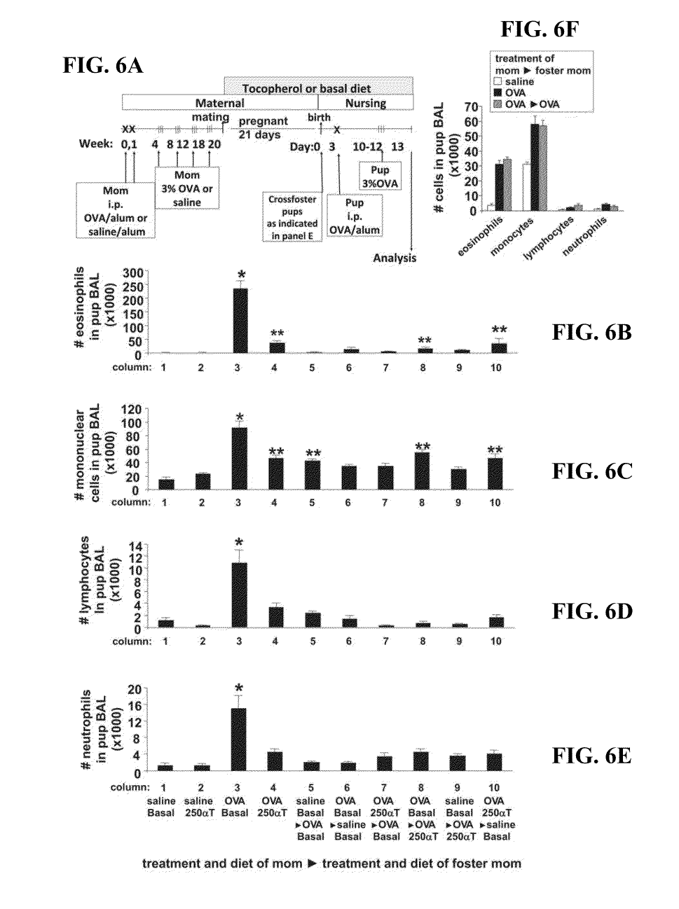

In FIG. 1, D-α-tocopherol supplementation was provided during pregnancy and lactation. To determine whether the maternal D-α-tocopherol supplementation during pregnancy or lactation was sufficient for inhibition of the allergic inflammation in the offspring, the neonates were cross-fostered on day 1 of birth as in the timeline in FIG. 6A. There was a contribution of the allergic mom during pregnancy and during lactation to the development of pup inflammation (FIG. 6, B-E, column 3 compared with columns 5 and 6) as previously described (44). Interestingly, cross-fostering pups from allergic mothers with 250 mg D-α-tocopherol/kg diet to allergic mothers with basal diet indicated that D-α-tocopherol supplementation of the allergic mother during pregnancy was sufficient to inhibit the development of the allergic response in the neonates (FIG. 6, B-E, column 7 compared with column 3). In addition, D-α-tocopherol supplementation during lactation reduced the allergic responses in the neonates (FIG. 6, B-E, column 8 compared with column 3). Pups from nonallergic female mice with basal diet, cross-fostered to allergic female mice with basal diet or 250 mg D-α-tocopherol/kg diet, did not develop allergic responses (FIG. 6, B-E, columns 5 and 9 compared with column 3). With basal diet, pups from allergic mothers cross-fostered to another allergic mother developed allergic responses (FIG. 6F).

### D-α-Tocopherol Supplementation During a Second Pregnancy of Allergic Female Mice Inhibited Development of Allergic Responses in the Second Litter of Pups

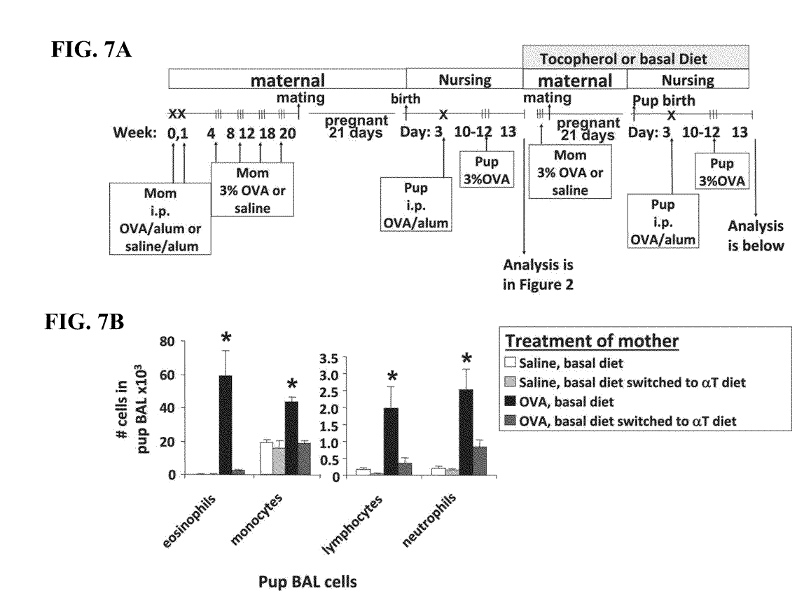

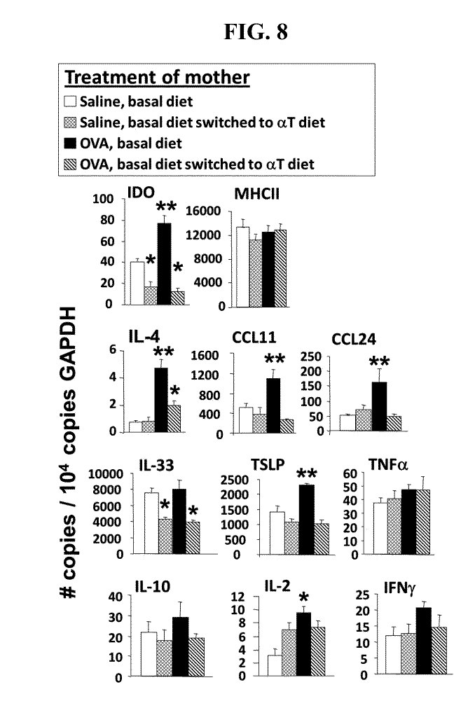

It was hypothesized that allergic moms that had a litter of allergic pups could be supplemented with D-α-tocopherol at the time of the second mating to inhibit development of allergic responses in subsequent litters of pups. Starting at the time of the second mating (timeline in FIG. 7A), the mothers, which were provided basal diet in the first mating, were provided in the second mating basal diet or switched to a diet supplemented with 500 mg D-α-tocopherol/kg diet. The offspring from allergic mothers that were supplemented with D-α-tocopherol at the time of the second mating had a >90% inhibition of BAL eosinophils in the OVA-challenged pups (FIG. 7B). Moreover, in OVA-challenged pups from allergic mothers, D-α-tocopherol reduced whole lung mRNA expression of several mediators of allergic inflammation: the cytokines IL-4, IL-33, and TSLP and the chemokines CCL11 and CCL24 (FIG. 8). At the time of collection, D-α-tocopherol had no effect on the cytokine TNF-α, the anti-inflammatory cytokine IL-10, Th1 cytokines (IL-2 and IFNγ), or MHCII in the whole lung of OVA-challenged pups from allergic mothers (FIG. 8). D-α-Tocopherol inhibition of IL-4, IL-33, and TSLP is consistent with α-tocopherol regulation of development of allergic inflammation. Because it is reported that DCs from pups of allergic moms but not control mothers transfer the risk for allergic responses to nonallergic pups (24) and because during allergic inflammation in adults lung dendritic cell expression of indolamine dioxygenase (IDO) promotes Th2 responses (83), we determined whether D-α-tocopherol supplementation regulated lung IDO expression. The OVA-challenged pups from allergic mothers from the studies in FIGS. 7A-B had elevated whole lung expression of IDO compared with OVA-challenged pups from saline-treated mothers (FIG. 8). Interestingly, D-α-tocopherol reduced lung tissue expression of IDO in pups from allergic mothers and saline mothers (FIG. 8). These data indicate that OVA challenge induced IDO expression and/or recruitment of cells expressing IDO and that this was inhibited by the D-α-tocopherol-supplemented diet. Thus, D-α-tocopherol supplementation of mothers during a second litter blocked allergic inflammation and several cytokines, chemokines, and IDO that regulated allergic inflammation.

D-α-Tocopherol Supplementation of Allergic Female Mice Reduces Numbers of CD11b+Dendritic Cell Subsets in the Fetal Liver and in Neonates

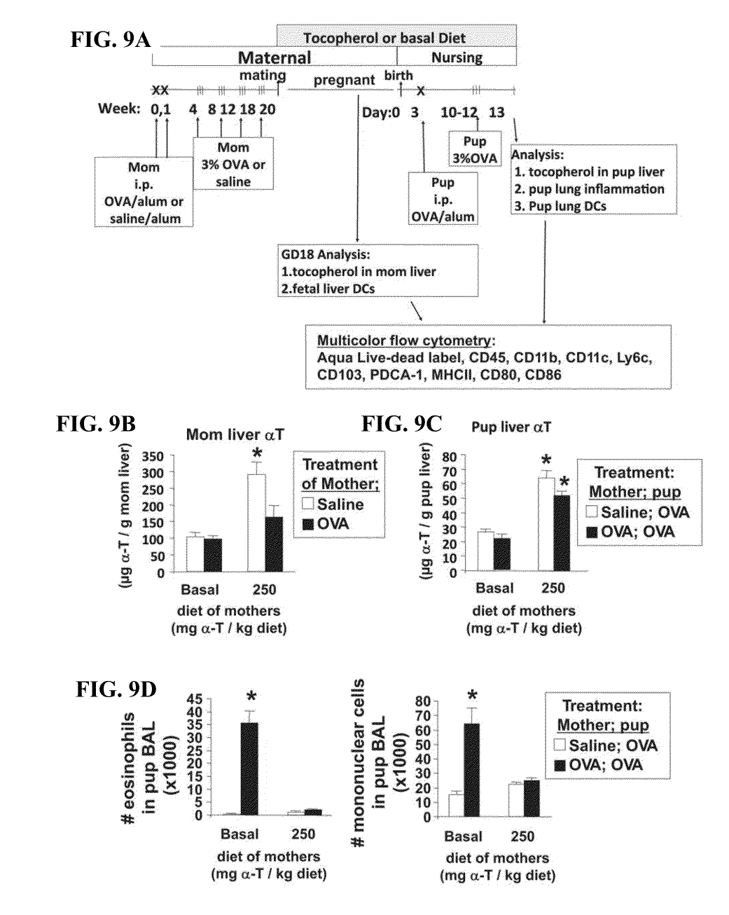

It is reported that DCs but not macrophages are critical for the development of the allergic responses in the pups from allergic mothers (24). Therefore, we determined whether D-α-tocopherol regulates the development of DCs in postnatal day 13 neonates and gestational day 18 fetuses from allergic mothers (FIG. 9A). It was also determined whether D-α-tocopherol-supplemented diets increased liver D-α-tocopherol concentrations in mothers and pups because, in the liver, D-α-tocopherol is loaded onto lipoproteins that then enter circulation for delivery to peripheral tissues. The D-α-tocopherol-supplemented diet significantly increased liver D-α-tocopherol in the saline-treated mothers threefold compared with basal diet controls (FIG. 9B). The OVA treatment reduced the D-α-tocopherol tissue concentrations in the D-α-tocopherol-supplemented mothers (FIG. 9B), which is consistent with reduced α-tocopherol levels in asthmatics (38, 39, 63, 64). Maternal D-α-tocopherol supplementation increased pup liver D-α-tocopherol 2.5-fold (FIG. 9C) and blocked allergic responses in the lung (FIG. 9D). These livers had <0.003 μg γ-tocopherol/g liver tissue for all groups (data not shown).

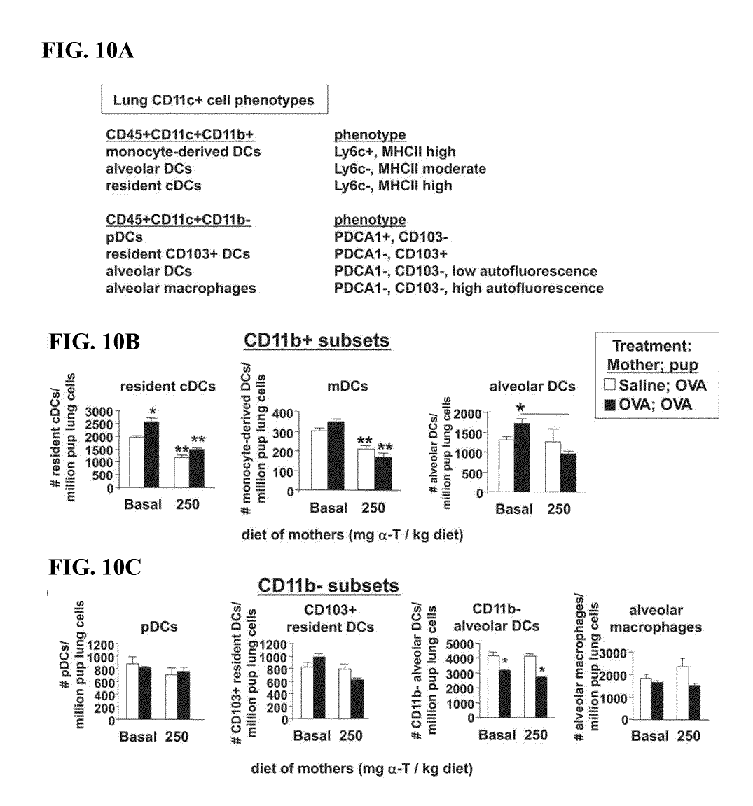

It was determined whether D-α-tocopherol regulates pup lung DCs. The cells of the postnatal day 13 neonate lungs or gestational day 18 fetal livers for the offspring outlined in FIG. 9A were dissociated and immunolabeled for cell membrane markers of lung dendritic cell subsets as previously described for the mouse lung (58). The flow cytometry analysis scheme for pup lung DCs is described in FIG. 2. There were CD11b+ and CD11b−dendritic cell subsets in the neonatal lungs, but D-α-tocopherol supplementation only altered the CD11b+ dendritic cell subsets so these two dendritic cell lineages are described here. Briefly, the following CD11b+ dendritic cell subsets were present in the postnatal day 13 OVA-challenged neonate lungs (FIG. 10A): resident DCs, monocyte-derived DCs (mDCs), and alveolar DCs. The following CD11b−cell subsets were present in the postnatal day 13, OVA-challenged neonate lungs (FIG. 10A): pDCs, resident CD103+DCs, alveolar DCs, and alveolar macrophages. In the postnatal day 13 OVA-challenged pups, maternal supplementation with D-α-tocopherol significantly reduced the lung tissue number of CD11c+CD11b+ subsets of DCs, including resident conventional DCs, mDCs, and CD11b+ alveolar DCs (FIG. 10B). In contrast to the CD11c+CD11b+DCs, D-α-tocopherol supplementation of the mothers did not alter the pup lung CD11c+CD11b−subsets of cells, including pDCs, resident CD103+DCs, CD11b−alveolar DCs, and alveolar macrophages (FIG. 10C). Pups from allergic mothers had reduced numbers of CD11c+CD11b−alveolar DCs compared with pups from nonallergic mothers, but there was no effect of maternal D-α-tocopherol supplementation (FIG. 10C). For CD11c+CD11b+DCs and CD11c+CD11b−DCs, the mean fluorescence intensity for MHCII, CD80, and CD86 was not different among the groups (data not shown).

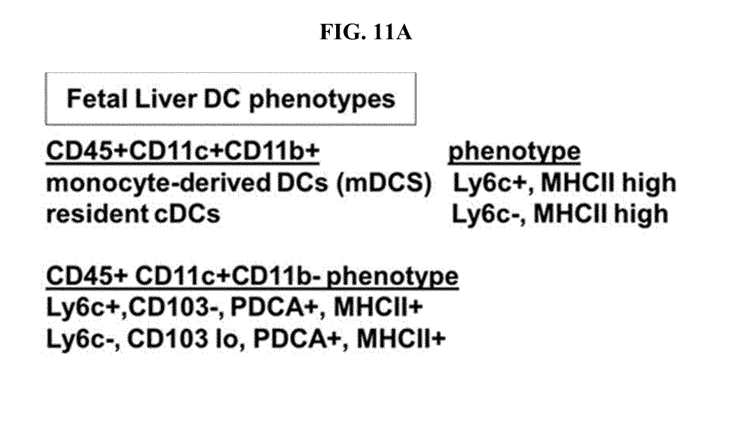

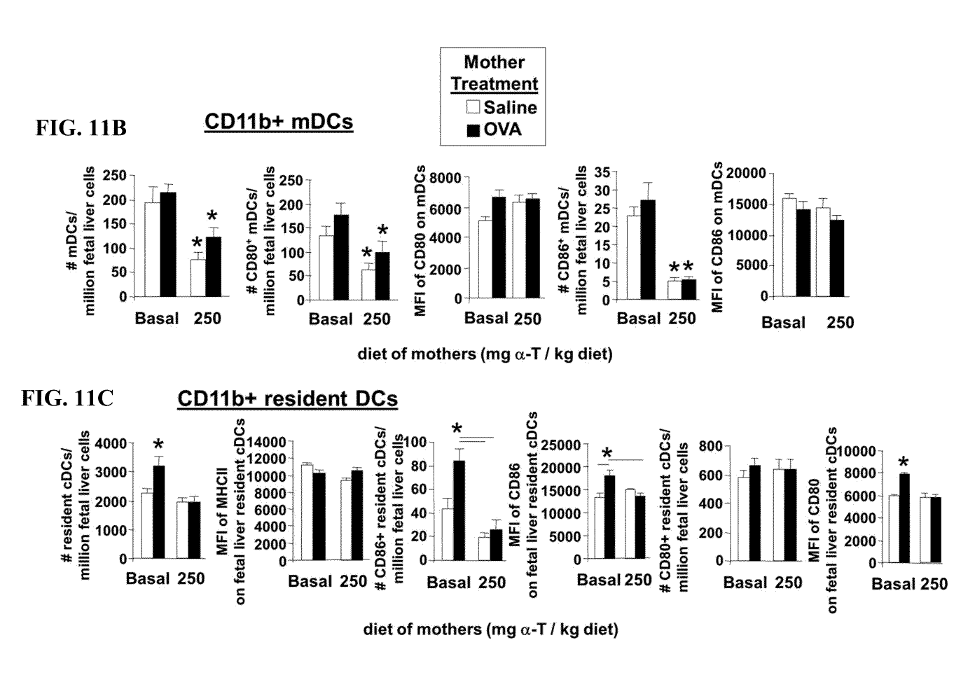

The fetal liver dendritic cell subsets were determined on gestational day 18 (FIG. 9A) because mouse fetal livers begin developing liver hematopoiesis about gestational day 16 and pups are born on gestational day 21. The fetal liver cells were immunolabeled for cell surface markers of DCs (FIG. 10A). The flow cytometry analysis scheme for fetal liver DCs is in FIG. 3. The following CD11c+CD11b+ dendritic cell subsets were present in the gestational day 18 fetal livers (FIG. 11A): mDCs and resident DCs. The following CD11c+CD11b−cell subsets were present in the gestational day 18 fetal livers (FIG. 11A): Ly6c+CD103−PDCA+MHCII+ cells and Ly6c−CD103lowPDCA+MHCII+ cells. Interestingly, D-α-tocopherol supplementation of allergic mothers reduced the number of CD11c+CD11b+DCs in gestational day 18 fetal livers, including those of the phenotype of mDCs (FIG. 11B) and phenotype of resident DCs (FIG. 11C). The CD11c+CD11b−subsets were not altered by maternal supplementation of D-α-tocopherol (data not shown).

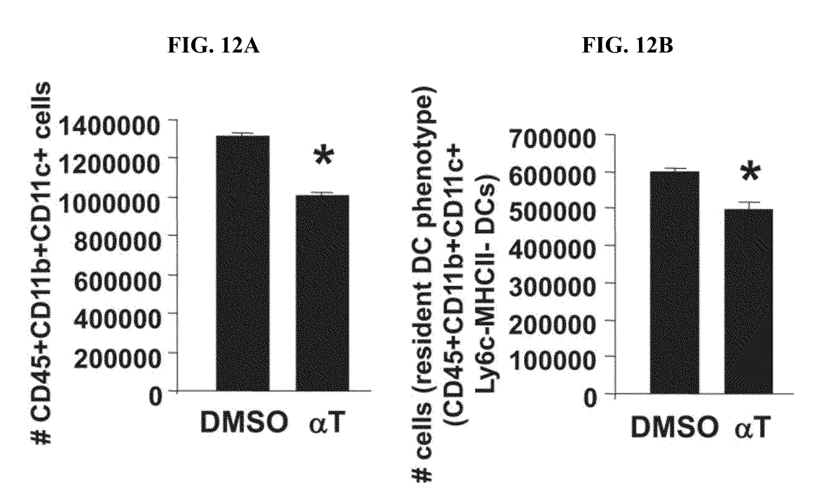

To determine whether D-α-tocopherol can directly regulate differentiation of DCs, we determined whether supplementation in vitro with D-α-tocopherol blocked development of bone marrow-derived CD11b+DCs. For these studies, bone marrow from 10-day-old neonates from mothers with basal diet was incubated with GM-CSF for 8 days in vitro in the presence of D-α-tocopherol or the solvent control DMSO. D-α-Tocopherol reduced the number of CD45+CD11b+CD11+DCs and the number of cells with resident DC phenotype (CD45+CD11b+CD11c+Ly6c−MHCII−DCs) (FIG. 12) without affecting the percent of viable cells in the culture (data not shown). In summary, D-α-tocopherol reduced the number of CD11b+CD11c+DCs in vitro, in the fetal liver and in the neonate.

### DISCUSSION

In this Example, the development of allergic inflammation in pups from allergic mothers was inhibited by supplementation of the mother with D-α-tocopherol during pregnancy and lactation. In the pups, there was a reduction in mediators of allergic inflammation, including IL-4, IL-33, TSLP, CCL11, and CCL24. In addition, after allergic mothers had a first litter of pups that responded to antigen challenge, D-α-tocopherol supplementation of these mothers during the second pregnancy prevented development of allergic inflammation in the second litter of pups that were challenged with antigen. The studies with cross-fostering at birth demonstrated that administration of D-α-tocopherol during pregnancy or during lactation was sufficient to inhibit development of allergic inflammation in the pups. D-α-Tocopherol did not affect body weight or lung weight, which is consistent with previous reports for D-α-tocopherol (7, 50, 52). D-α-Tocopherol supplementation of the mothers inhibited generation of CD11c+CD11b+DCs in the fetal liver and in antigen-challenged pup lungs. D-α-Tocopherol has, at least, a direct effect on dendritic cell development because D-α-tocopherol inhibited generation of CD11c+CD11b+ bone marrow-derived DCs in vitro. In contrast, D-α-tocopherol did not alter CD11c+CD11b−DCs in vivo or in vitro, indicating specificity for D-α-tocopherol regulation of dendritic cell differentiation to CD11c+CD11b+DCs. There was no effect of D-α-tocopherol on the level of expression of MHCII, CD80, or CD86 by DCs. This Examples demonstrates that maternal D-α-tocopherol supplementation of allergic mothers reduces development of allergic responses in offspring and regulates numbers of select dendritic cell subsets.

The prevalence of allergic diseases has dramatically increased in the last 40 years (27, 73, 76), suggesting that there are changes in environmental factors. Allergic diseases originate as complex environmental and genetic interactions that may start early in life (49), a time when the airways and the immune system are developing. It is suggested that in utero and early exposures to environmental factors are critical for risk of allergic disease (9). In reports examining human maternal and paternal asthma associations with development of allergies in offspring, most associations are with maternal asthma (46). In animal studies, allergen challenge of the mother predisposes the offspring to responses to suboptimal doses of allergens and, as in humans (46), the offspring's responses are not specific to the allergen that the mother had been exposed (26, 28-30, 32, 33, 36). The sensitization to allergens and allergic responses are dependent on DCs, which produce regulatory cytokines (72, 80). Interestingly, DCs, but not macrophages, from offspring of allergic mothers transfer the risk for allergies to naïve neonates, indicating a functional change in neonatal DCs during development (24). Maternal exposure to environmental factors, including high-fat diets, can alter neonatal hematopoietic or metabolic function (14, 25, 35, 47, 55, 61, 75, 82). In our studies, maternal supplementation with D-α-tocopherol inhibited development of CD11c+CD11b+DCs in the fetus and in neonates.

Dietary tocopherols are taken up from the intestine (62) and transported through the lymph to the blood and then to the liver. In the liver, α-tocopherol is preferentially transferred, over other tocopherol isoforms, to lipoproteins by the liver αTTP (11, 45, 59, 68, 81). αTTP is also expressed by trophoblast, fetal endothelium, and amnion epithelium of the placenta (54). αTTP transfers α-tocopherol to low-density lipoprotein (LDL) and high-density lipoprotein (HDL) particles that then enter the blood (23). LDL or HDL with tocopherols are taken up by cells by plasma phospholipid transfer protein, scavenger receptors, or the lipoprotein lipase pathway (21). It is reported that basal levels of α-tocopherol are required for placentation (37,54). In our studies, maternal supplementation with D-α-tocopherol raised the maternal liver α-tocopherol level 3-fold and the pup liver α-tocopherol 2.5-fold, consistent with the fold tocopherol changes in human and mouse tissues after supplementation (2, 7, 19, 20, 50, 51). This increase in maternal D-α-tocopherol inhibited allergic responses in neonates and inhibited development of CD11c+CD11b+ dendritic cell subsets in utero and in the neonate.

The numbers of neonatal lung dendritic cell subsets in our studies were consistent with other reports of proportions of DCs in lungs of neonatal mice and adult mice. Reports indicate that, for neonatal mice, there are about 1×10⁵ CD11c+MHCII+ cells/pup lung in 4- to 11-day-old neonates (85), and there are about 5×10⁴ pDCs/pup lung in 15-day-old neonates (4). Consistent with this, in our studies in which there were 20×10⁶ lung cells/10-day-old C57BL/6 pup lung (data not shown), there were 2×10⁵CD11c+ cells/pup lung and 2×10⁴ pDCs/pup lung. In addition, the proportions of several dendritic cell subsets in pups in our studies were similar to reports of adult lungs. Briefly, the ratio of pDC/cDC is reported as about 0.2-0.3 in the OVA-challenged adult mouse lung (41). In the 10-day-old pups in our studies, the lung pDC-to-cDC ratio was also 0.3 in the lungs of OVA-challenged pups from allergic mothers with basal tocopherol diet. Interestingly, with D-α-tocopherol supplementation of allergic mothers, there was a decrease in numbers of pup lung cDC, resulting in an increase in the pDC-to-cDC ratio to 0.53. Also in adult mice, about 8% of lung CD11c+ cells are pDCs (60) and 9% of lung CD11c+DCs are CD103+(57). Likewise, in the 10-day-old C57BL/6 pup lungs in our study, 8% of lung CD11c+ cells were pDCs and 9.7% of CD11c+DCs were CD103+. Thus, proportions of DCs in the neonatal mouse lungs in our studies were similar to the few reports for the neonatal mouse lung and to reports for the adult mouse lung.

In our studies, maternal D-α-tocopherol supplementation decreased the number of pup lung CD11b+ dendritic cell subsets but not the number of pup lung CD11b−dendritic cell subsets. This specificity of α-tocopherol regulation of the CD11b+ dendritic cell subsets suggests that tocopherols may regulate signals for dendritic cell differentiation of this DC subset or regulate signals for expression of CD11b. The generation of CD11b+DCs is regulated by signals and several transcription factors that bind the CD11b promoter (74). Some signals that regulate expression of CD11b on DCs include GM-CSF (86), ERK5 (78), aldehyde dehydrogenase (86), and retinoic acid (5, 40, 66, 67, 86). The CD11b promoter has binding sites for the transcription factors Sp1, Vav1/PU.1, ets, AP-2, and RAR (12, 31). Whether α-tocopherol regulates GM-CSF signals or regulates transcription factors that activate the CD11b promoter in DCs is not known and is under investigation.

In DCs, signaling through protein kinase C and NF-kB induces IDO expression (56). The IDO pathway can function to inhibit Th1 responses (84), whereas IDO has an opposing role in which it increases Th2 immune responses (84). Consistent with this, IDO-deficient adult mice have fewer mature DCs in draining lymph nodes, reduced OVA-induced inflammation, and lower airway hyperresponsiveness (83). It is reported that, in patients, serum IDO is increased during seasonal allergen exposure and that IDO expression is induced by the high-affinity receptor for IgE, FcεRI (77). It is also reported that, during pregnancy, IDO promotes tolerance toward the fetus (43). In our studies, pups from allergic mothers had elevated OVA-induced lung IDO expression, and this was reduced by maternal D-α-tocopherol supplementation without affecting the number of pups or gender distribution. Thus the OVA challenge induced lung IDO expression and/or recruitment of cells expressing IDO in the pup lung, and this was inhibited by maternal supplementation with D-α-tocopherol.

### Example 2

### Maternal γ-Tocopherol (γT) Supplementation Elevates Development of Allergic Inflammation in Offspring of Allergic Mothers

Example 1 above described that maternal supplementation with alpha-tocopherol (αT) reduced development of dendritic cell subsets and allergic responses in offspring of allergic female mice. In this Example, allergic female mice were supplemented with γT during pregnancy/lactation. Then, offspring were given a suboptimal allergen challenge. γT supplementation of allergic mothers elevated pup responses to allergen challenge. There were increased numbers of pup lung eosinophils, inflammatory mediators, and CD11b expressing but not CD11b-subsets of CD11c dendritic cells. There were elevated numbers of IRF4+CD11b+CD11c+ expressing dendritic cells, a dendritic cell subset critical for development of allergic responses. There were also fewer pups from γT supplemented allergic mothers. In conclusion, maternal αT supplementation reduced and maternal γT supplementation increased development of CD11b+CD11c+ dendritic cells and allergic responses in offspring from allergic mothers.

### Animals—

C57BL/6 mice were obtained from Jackson Laboratories.

### Experimental Asthma Protocol and Tocopherol Administration.

C57BL/6 female mice were sensitized with OVA/alum and challenged with OVA (150 μg) as in the timeline below. Then, the cages were supplied with basal diet (45 mg γ-tocopherol/kg diet) or γ-tocopherol supplemented diets (250 mg γ-tocopherol/kg diet) and males were introduced for breeding. The pups were administered a suboptimal dose of OVA/alum (i.e., one i.p. with OVA/alum and 3 challenges for 10 min with 3% OVA as previously described (Am J Respir Cell Mol Biol 2011. 44: 285 and Am J PhysiolLung Cell MolPhysiol. 2014, 15; 307:L482-96; both of which are herein incorporated by reference in their entireties).

### Results

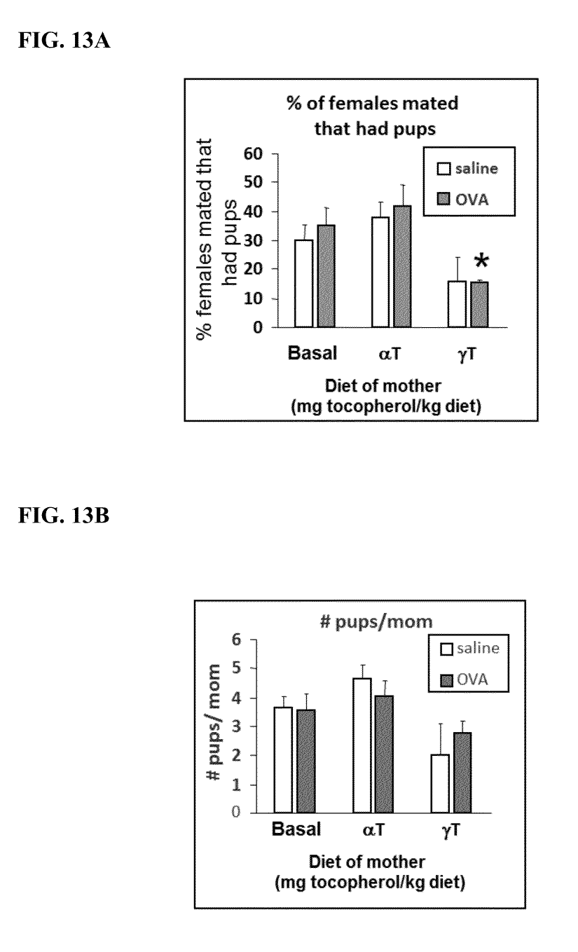

FIGS. 13A-B show that maternal γ-tocopherol-supplemented diet reduced the number of allergic mothers with pups. FIG. 13A shows the percentage of mated females that had pups, and FIG. 13B shows the number of pups per mom.

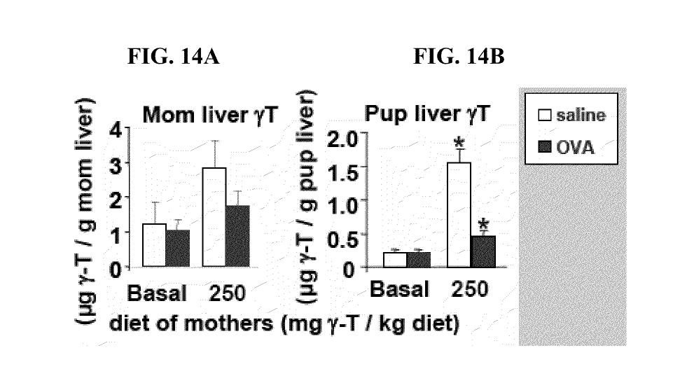

FIGS. 14A-B show maternal γ-tocopherol-supplemented diet elevates tissue γ-tocopherol in mothers (FIG. 14A) and pups (FIG. 14B).

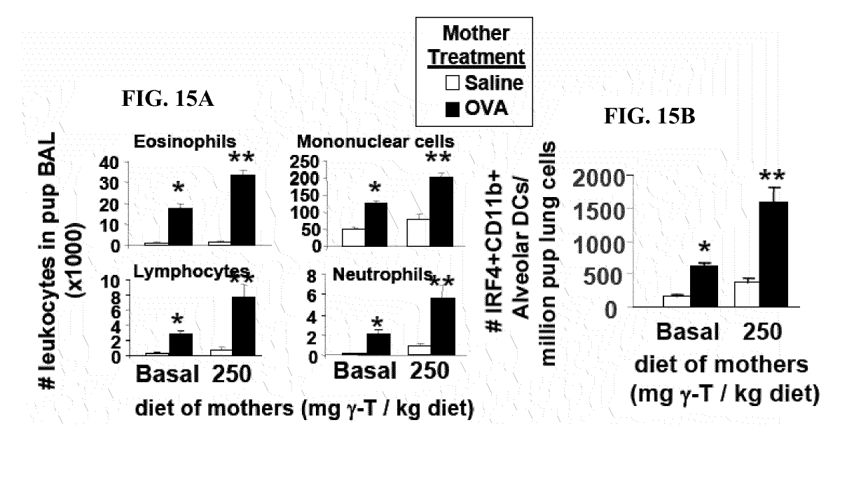

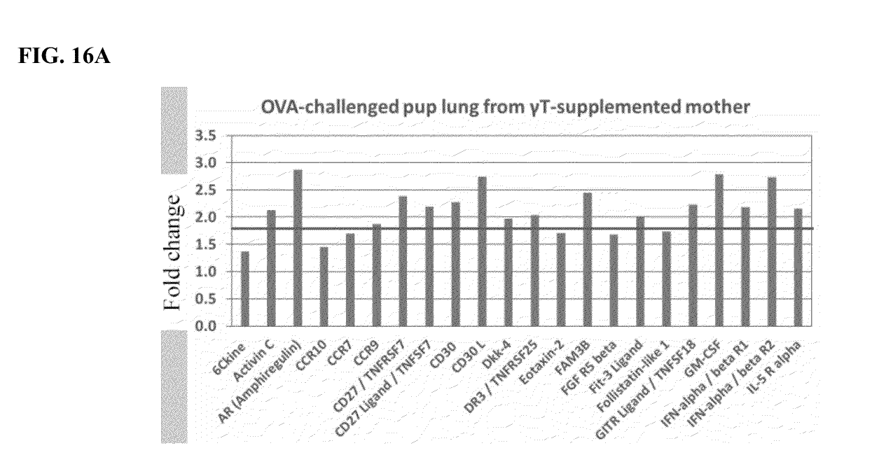

FIGS. 15A-B show that a maternal γ-tocopherol-supplemented diet increases the number of BAL leukocytes and number of IRF4+CD11c+CD11b+ dendritic cells in OVA-challenged pups from allergic mothers. FIG. 15A shows the number of leukocytes in pup BAL, and FIG. 15B shows the number of IRFA+CD11b+ alveolar DCs per million pup lung cells.

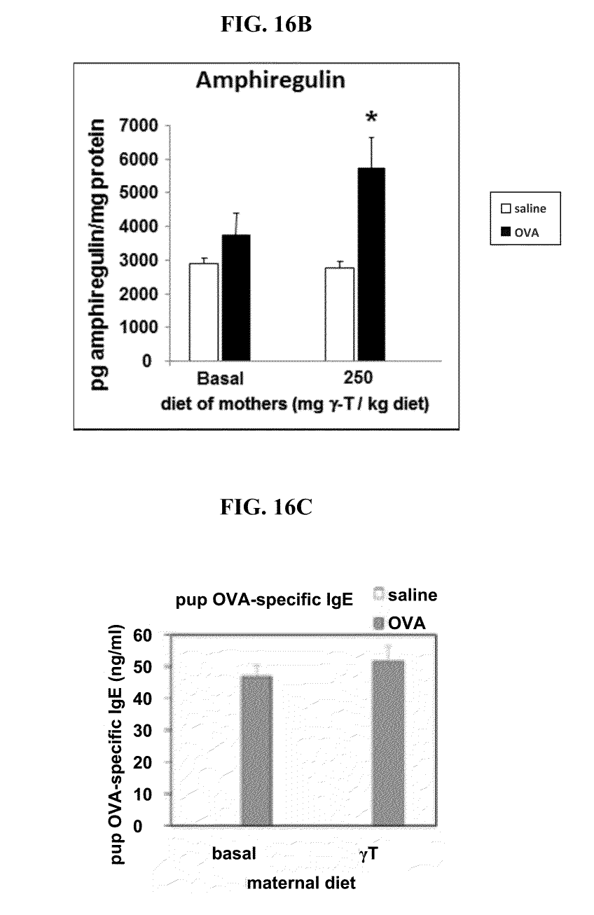

FIGS. 16A-C show maternal γ-tocopherol diet increases inflammatory mediators in OVA-treated pup lungs. FIG. 16A shows the results from RayBiotech protein array (308 proteins) with 2 hour minced lung culture supernatants. Shown is fold change of protein from lungs of OVA-stimulated pups from γT-supplemented allergic mothers compared to lungs from allergic mothers fed basal diet. Above the red line is considered significant in the array. FIG. 16B shows an ELISA from culture supernants in panel A. FIG. 16C shows an ELISA for OVA-specific IgE in serum from pups.

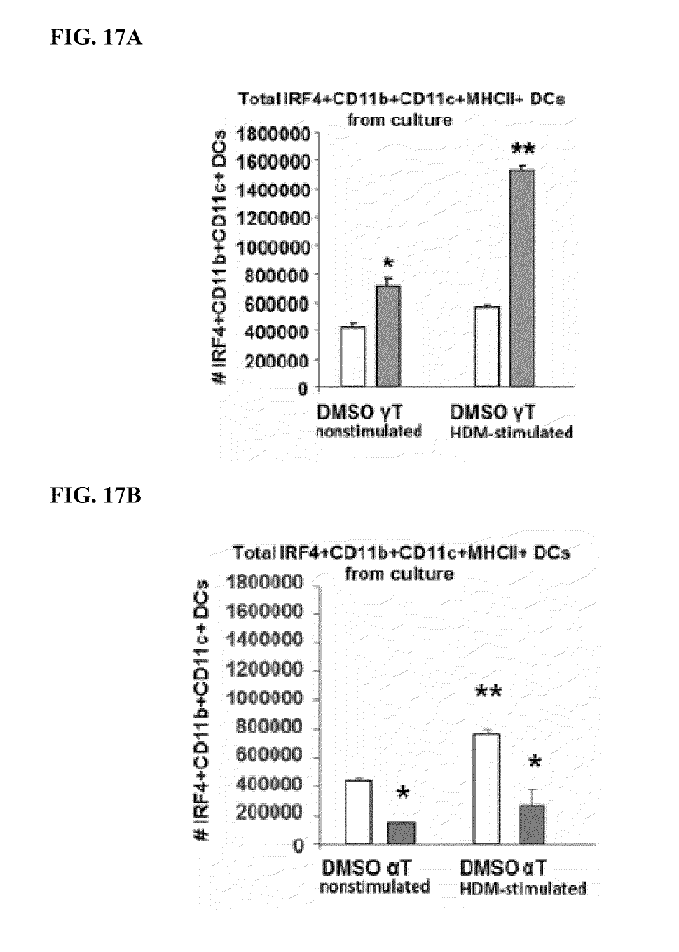

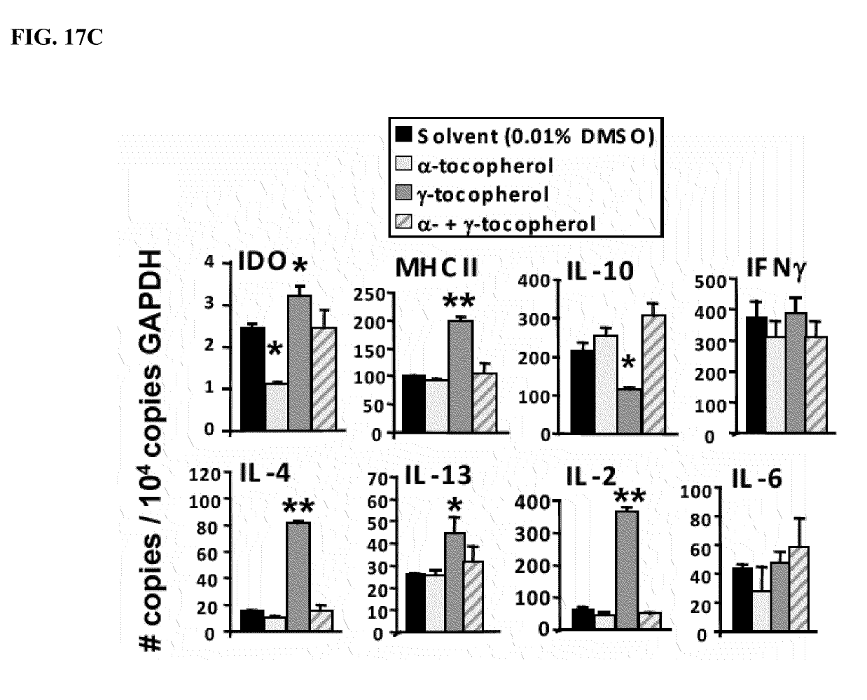

FIGS. 17A-C show α-T and γ-T have opposing effects in regulation of DC development and function in vitro. FIGS. 17A and B show results from bone marrow from postnatal day 10 neonate (with basal diet) that was cultured with GM-CSF and with 80 μM αT (FIG. 17B) or 2 μM γT (FIG. 17A) for 8 days and analyzed for DC subsets by immunolabeling/flow cytometry. *, p<0.05 compared to DMSO group. **, p<0.05 compared to indicated group. FIG. 17 C shows results from bone marrow-derived DCs that were co-cultured 48 hours with purified CD4+ T cells with and without 80 μM α-T or 2 μM γ-T, or both tocopherols. Expression by qPCR. There was no effect on cell viability (not shown). n=3. **, p<0.05 compared to all other groups. *, p<0.05 compared to DMSO group.

### REFERENCES

1. Abdala-Valencia et al., Nutrients 5: 4347-4363, 2013.
2. Abdala-Valencia et al., PLoS ONE 7: e41054, 2012.
3. Abdala-Valencia et al., Am J Physiol Lung Cell Mol Physiol 292: L1111-L1125, 2007.
4. Al-Garawi et al., Mucosal Immunol 4: 682-694, 2011.
5. Beijer et al., Immunology 142: 39-45, 2014.
6. Berdnikovs et al., Am J Physiol Lung Cell Mol Physiol 304: L240-L249, 2013.
7. Berdnikovs et al., J Immunol 182: 4395-4405, 2009.
8. Betti et al., J Nutr Biochem 22: 60-70, 2011.
9. Bousquet J, et al., Allergy 66: 596-604, 2011.
10. Bousquet et al., Bull World Health Organ 83: 548-554, 2005.
11. Brigelius-Flohe et al., FASEB J 13: 1145-1155, 1999.
12. Brugnoli et al., Exp Cell Res 316: 38-47, 2010.
13. Bryce et al., J Allergy Clin Immunol 112: 149-158, 2003.
14. Burke et al., J Lipid Res 50: 1146-1155, 2009.
15. Cook-Mills J M. Eosinophil-endothelial cell interactions. Eosinophils in health and disease. In: Eosinophils in Health and Disease, edited by Lee J JaR. New York, N.Y.: Elsevier, 2012, p. 139-153.
16. Cook-Mills et al., J Clin Cell Immunol 4: 1000137, 2013.
17. Cook-Mills et al., Am J Respir Crit Care Med 188: 279-284, 2013.
18. Cook-Mills et al., Antioxid Redox Signal 15: 1607-1638, 2011.
20. Cook-Mills et al., Endocr Metab Immune Disord Drug Targets 10: 348-366, 2010.
21. Desrumaux et al., FASEB J 13: 883-892, 1999.
22. Dow et al., Am J Respir Crit Care Med 154: 1401-1404, 1996.
23. Fechner et al., Biochem J 331: 577-581, 1998.
24. Fedulov et al., Am J Respir Cell Mol Biol 44: 285-292, 2011.
25. Fedulov et al., Am J Respir Cell Mol Biol 38: 57-67, 2008.
26. Fedulov et al., Am J Reprod Immunol 58: 120-128, 2007.
27. Friebele E., Environ Health Perspect 104: 22-25, 1996.
28. Hamada et al., J Immunol 170: 1683-1689, 2003.
29. Herz et al., Int Arch Allergy Immunol 124: 193-196, 2001.
30. Herz et al., Am J Respir Crit Care Med 162: S62-65, 2000.
31. Hickstein et al., Proc Natl Acad Sci USA 89: 2105-2109, 1992.
32. Hubeau et al., Am J Pathol 168: 1931-1939, 2006.
33. Hubeau et al., J Immunol 178: 1477-1487, 2007.
34. Hunter et al., Lipids 42: 97-108, 2007.
35. Izzotti et al., FASEB J 17: 1127-1129, 2003.
36. Jarrett et al., Nature 280: 145-147, 1979.
37. Jishage et al., Biol Reprod 73: 983-987, 2005.
38. Kalayci et al., Turk J Peds 42: 17-21, 2000.
39. Kelly et al., Lancet 354: 482-483, 1999.
40. Klebanoff et al., J Exp Med 210: 1961-1976, 2013.
41. Kool et al., J Immunol 183: 1074-1082, 2009.
42. Lass et al., Free Rad Biol Med 26: 1375-1382, 1999.
43. Le et al., Curr Allergy Asthma Rep 6: 27-31, 2006.
44. Leme et al., J Immunol 176: 762-769, 2006.
45. Leonard et al., Am J Clin Nutr 75: 555-560, 2002.
46. Lim et al., Am J Reprod Immunol 61: 1-10, 2009.
47. Lim et al., PLoS ONE 5: e10134.
48. Marchese et al., Respir Res 15: 31, 2014.
49. Martinez F D, Eur Respir J 29: 179-184, 2007.
50. McCary et al., J Immunol 186: 3674-3685, 2011.
51. McCary et al., Biochem J 441: 189-198, 2012.
52. Meydani et al., Lipids 22: 345-350, 1987.
53. Meydani et. al., J Nutr 118: 1245-1252, 1988.
54. Muller-Schmehl et al., Free Radic Res 38: 413-420, 2004.
55. Odaka et al., Obesity 18: 1688-1694, 2010.
56. Ogasawara et al., J Cell Biochem 108: 716-725, 2009.
57. Plantinga et al., Immunity 38: 322-335, 2013.
58. Plantinga et al., Eur J Immunol 40: 2112-2118, 2010.
59. Podda et al., J Lipid Res 37: 893-901, 1996.
60. Raymond et al., J Allergy Clin Immunol 124: 1333-1342, 2009.
61. Rebholz et al., Am J Physiol Endocrinol Metab 302: E449-E457, 2012.
62. Reboul et al., J Biol Chem 281: 4739-4745, 2006.
63. Schunemann et al., Am J Respir Crit Care Med 163: 1246-1255, 2001.
64. Shvedova et al., Tox Appl Pharm 132: 72-81, 1995.
65. Smit et al., Proc Nutr Soc 58: 309-319, 1999.
66. Stock et al., Eur J Immunol 43: 1441-1444, 2013.
67. Tang et al., Acta Biochim Biophys Sin (Shanghai) 41: 588-593, 2009.
68. Traber et al., Am J Clin Nutr 49: 517-526, 1989.
69. Traber et al., Free Radic Biol Med 38: 773-785, 2005.
70. Troisi et al., Am J Respir Crit Care Med 151: 1401-1408, 1995.
71. Umegaki et al., Int J Vitamin Nutr Res 67: 149-154, 1997.
72. van Rijt et al., Clin Exp Allergy 35: 1125-1134, 2005.
73. van Schayck et al., Eur Respir J 26: 647-650, 2005.
74. Vander et al., Nat Immunol 15: 161-167, 2014.
75. Vanhees et al., FASEB J 25: 797-807, 2011.
76. Vollmer et al., Am J Respir Crit Care Med 157: 1079-1084, 1998.
77. von Bubnoff et al., Clin Exp Allergy 34: 1056-1063, 2004.
78. Wang et al., J Cell Physiol 229: 856-867, 2014.
79. Weiss S T. Ciba Fndn Symp 206: 244-257, 1997.
80. Williams et al., Nat Commun 4: 2990, 2013.
81. Wolf G. Nutr Rev 64: 295-299, 2006.
82. Woollett et al., Am J Clin Nutr 82: 1155-1161, 2005.
83. Xu et al., Proc Natl Acad Sci USA 105: 6690-6695, 2008.
84. Xu et al., Immunol Lett 121: 1-6, 2008.
85. Yamaguchi et al., J Virol 86: 10456-10461, 2012.
86. Yokota et al., Int Immunol 21: 361-377, 2009.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A method of preventing, or reducing the severity of, a condition in a neonate, infant, or child comprising:
administering a composition to a mother of a neonate, infant, or child: 1) prior to birth of said neonate, infant, or child, and/or 2) during a time period wherein said mother is breast feeding said neonate, infant, or child;
wherein said composition comprises at least 5 international units (IU's) of tocopherol, wherein at least 98% of all of said tocopherol in said composition is unmodified natural d-alpha-tocopherol, and wherein less than 2% of all of said tocopherol in said composition is gamma-tocopherol, and
wherein said administering prevents, or reduces the severity of, an inflammatory condition in said neonate, infant, or child.

2. The method of claim 1, wherein said condition premature fetal lung disease, full term pulmonary distress, childhood asthma, or an allergic condition.

3. The method of claim 1, wherein said mother has at lease one type of allergy.

4. The method of claim 3, wherein said method further comprising, prior to said administering, testing said mother for at lease one type of allergy.

5. The method of claim 1, wherein at least 99% of all of said tocopherol in said composition is unmodified natural d-alpha-tocopherol, and wherein less than 1% of all of said tocopherol in said composition is gamma-tocopherol.

6. The method of claim 1, wherein at least 99.9% of all of said tocopherol in said composition is unmodified natural d-alpha-tocopherol, and wherein less than 0.1% of all of said tocopherol in said composition is gamma-tocopherol.

7. The method of claim 6, wherein said gamma-tocopherol is undetectable or nearly undetectable in said composition.

8. The method of claim 1, wherein said administering to said mother is daily during the course of at least one trimester of the pregnancy of said mother.

9. The method of claim 1, further comprising, after said birth of said neonate, infant, or child, administering said composition to said neonate, infant, or child.

10. The method of claim 1, wherein said composition is administered daily to said subject for a period selected from: about 1 week, about 2 weeks, about 3 weeks, about 1 month, about 2-3 months, about 3-6 months, about 6-9 months, about 12-18 months, about 18 to 24 months, or about 24 to 30 months.

11. The method of claim 1, wherein said composition comprises a prenatal pill suitable for daily administration, wherein said prenatal pill comprises: 1) said tocopherol, and 2) at least one additional ingredient selected from: A) at least 300 μg of folic acid, B) at least 20 mg of iron, and C) at least 125 mg of calcium.

12. The method of claim 11, wherein said prenatal pill comprises said tocopherol, said folic acid, said iron, and said calcium.

13. The method of claim 11, wherein said at least 300 ug of folic acid is at least 600 μg of folic acid.

14. The method of claim 11, wherein said at least 20 mg of iron is at least 27 mg of iron.

15. The method of claim 11, wherein said at least 125 mg of calcium is at least 900 mg of calcium.

16. The method of claim 1, wherein said tocopherol is present in said composition at at least 10 international units (IUs).

17. A composition comprising:
a) at least 5 international units (IUs) of tocopherol, wherein at least 98% of all of said tocopherol in said composition is unmodified natural d-alpha-tocopherol, and wherein less than 2% of all of said tocopherol in said composition is gamma-tocopherol;
b) at least 300 μg of folic acid,
c) at least 20 mg of iron, and
d) at least 125 mg of calcium.

18. The composition of claim 17, wherein said composition is formulated in a pre-natal tablet or pre-natal pill, wherein said at least 8 IUs of tocopherol is at least 25 IUs of tocopherol, wherein said at least 300 μg of folic acid is at least 600 μg of folic acid, wherein said at least 20 mg of iron is at least 27 mg of iron, and wherein said at least 125 mg of calcium is at least 800 mg of calcium.

19. The composition of claim 17, further comprising at least one of the following: 200 μg of iodine, at least 3000 IUs of vitamin A, at least 60 mg of vitamin C, at least 300 IUs of vitamin D, at least 1.25 mg of vitamin B1, at least 1.5 mg of vitamin B2, at least 15 mg of vitamin B3, at least 2.0 mg of vitamin B6, at least 3 μg of vitamin B12, at least 10 mg of zinc, at least 200 μg of biotin, at least 8 mg of pantothenic acid, and at least 2 mg of copper.

20. A system comprising:
a) said composition of claim 17 formulated as a tablet, and
b) a pill comprising DHA (Docosahexaenoic acid) and/or EPA (Eicosapentaenoic acid).
